(19) United States
(12) Patent Application Publication
(10) Pub. No.: US 2026/0276653 A1
(43) Pub. Date: Sep. 17, 2026

(54) DIAGNOSIS, MONITORING OR PROGNOSIS OF TYPE 2 DIABETES, IMPAIRED GLUCOSE TOLERANCE OR PREDIABETES

(71) Applicants: SERVIZO GALEGO DE SAÚDE, Santiago de Compostela (ES); FUNDACIÓN PÚBLICA GALEGA INSTITUTO DE INVESTIGACIÓN SANITARIA DE SANTIAGO DE COMPOSTELA, Santiago de Compostela (ES); UNIVERSIDADE DE SANTIAGO DE COMPOSTELA, Santiago de Compostela (ES)

(72) Inventors: Manuela ALONSO SAMPEDRO, Santiago de Compostela (ES); Arturo GONZÁLEZ QUÍNTELA, Santiago de Compostela (ES); Francisco GUDE SAMPEDRO, Santiago de Compostela (ES)

(73) Assignees: SERVIZO GALEGO DE SAÚDE, Santiago de Compostela (ES); FUNDACIÓN PÚBLICA GALEGA INSTITUTO DE INVESTIGACIÓN SANITARIA DE SANTIAGO DE COMPOSTELA, Santiago de Compostela (ES); UNIVERSIDADE DE SANTIAGO DE COMPOSTELA, Santiago de Compostela (ES)

(21) Appl. No.: 19/163,094

(22) PCT Filed: Mar. 11, 2024
(86) PCT No.: PCT/EP2024/056392

§ 371 Date: Sep. 8, 2025

(30) Foreign Application Priority Data
Mar. 9, 2023 (EP) 23382220.4

(51) Int. Cl. G01N 33/68

(52) U.S. Cl. CPC G01N 33/6893; G01N 2333/4716; G01N 2333/4728; G01N 2333/78; G01N 2800/042; G01N 2800/50; G01N 2800/52

(57) ABSTRACT

The present invention refers to an in vitro method for the diagnosis, monitoring or prognosis of IGT/diabetes or prediabetes; or for screening or selecting patient candidates to be subjected to OGTT, which comprises assessing the amount of a protein, selected from the group consisting of: Retinol-Binding Protein 4 (Rbp), Alpha-1-acid glycoprotein (Agp), fibronectin (FN), and/or Complement factor H (CFH) which is bound to glycosaminoglycans (GAGs).

### TECHNICAL FIELD

The present invention refers to the medical field. Particularly, the present invention refers to an in vitro method for the diagnosis, monitoring or prognosis of IGT/diabetes or prediabetes; or for screening or selecting patient candidates to be subjected to oral glucose tolerance test (OGTT), which comprises assessing the amount of a protein, selected from the group consisting of: Retinol-Binding Protein 4 (Rbp), Alpha-1-acid glycoprotein (Agp), fibronectin (FN) and/or Complement Factor H (CFH), which is bound to glycosaminoglycans (GAGs).

### STATE OF THE ART

Diabetes mellitus is a complex, chronic illness characterized by elevated blood glucose levels that occurs when there is cellular resistance to insulin action, pancreatic B-cells do not produce sufficient insulin, or both. The number of cases and diabetes prevalence have greatly increased in recent decades; consequently, it is considered an important public health problem, one of four priority noncommunicable diseases (NCDs) targeted for action by world leaders.

Globally, an estimated 422 million adults were living with diabetes in 2014, compared to 108 million in 1980. The global prevalence (age-standardized) of diabetes has nearly doubled since 1980, rising from 4.7% to 8.5% in the adult population. The most common form of diabetes mellitus, type 2 diabetes (T2D), represents approximately 90% of all cases worldwide. T2D occurs more often in middle-aged and elderly adults, and its cause is multifactorial. However, its incidence has increased in children and young adults due to obesity, sedentary lifestyle, and inadequate nutrition. This high incidence is also accompanied by an estimate underdiagnoses prevalence of more than 50% worldwide.

Poor blood glucose control can result in long-term micro- and macrovascular complications such as nephropathy, retinopathy, neuropathy, and cardiovascular disease. Diabetes caused 1.5 million deaths in 2012. Higher-than-optimal blood glucose caused an additional 2.2 million deaths, by increasing the risks of cardiovascular and other diseases. Forty-three percent of these 3.7 million deaths occur before the age of 70 years.

Individuals with diabetes require continuous medical care, including pharmacological intervention as well as lifestyle and dietary changes. Diabetes and its complications bring about substantial economic loss to people with diabetes and their families and to health systems and national economies through direct medical costs and loss of work and wages.

According to the “Standards of Medical Care in Diabetes” published by the American Diabetes Association (ADA) and the World Health Organization (WHO) guidelines, diabetes may be diagnosed based on the concentration of plasma glucose-either fasting plasma glucose (FPG) or 2-hour plasma glucose during a 75 g oral glucose tolerance test (OGTT)- or based on glycated hemoglobin A1c (A1C) concentration. Prediabetes is an intermediate hyperglycemic state in which glycemic markers such as blood glucose and A1C are above the threshold considered healthy but below the diagnostic criteria for diabetes. This state constitutes a high risk for the development of diabetes and complications associated with the loss of glycemic control. The diagnostic reference values for prediabetes and diabetes have not been universally standardized. However, the vast majority of clinical guidelines are based on the WHO and ADA criteria (Table 1). In the absence of unequivocal hyperglycemia, diagnosis requires two abnormal test results from the same sample or in two separate test samples. In a patient with classic symptoms of hyperglycemia or hyperglycemic crisis, the diagnosis is a random plasma glucose≥200 mg/dL [American Diabetes Association. Standards of medical care in diabetes—2022. Diabetes Care, 2022; 45: S1. «Global Report on Diabetes». World Health Organization. 2016].

TABLE 1
Glycemic state diagnostic reference values

| | FPG | A1C | OGTT, 2-h PG |
|---|---|---|---|
| Normoglycemia | | | NGT |
| ADA | <100 mg/dL | <5.7% | <140 mg/dL |
| WHO | <110 mg/dL | | |
| Prediabetes | | | IGT |
| ADA | 100-125 mg/dL | 5.7-6.4% | 140-199 mg/dL |
| WHO | 110-125 mg/dL | | |
| Diabetes | | | |
| ADA | ≥126 mg/dL | ≥6.5% | ≥200 mg/dL |
| WHO | | | |

FPG, fasting plasma glucose (fasting is defined as no caloric intake for at least 8 h);
A1C, glycated hemoglobin A1c (the test should be performed in a laboratory using a method that is NGSP certified and standardized to the DCCT assay);
OGTT, oral glucose tolerance test (the test should be performed using a glucose load containing the equivalent of 75 g anhydrous glucose dissolved in water);
2-h PG, plasma glucose 2-h after OGTT;
NGT, normal glucose tolerance;
IGT, impaired glucose tolerance;
ADA, American Diabetes Association;
WHO, World Health Organization.

It should be noted that the screening tests do not necessarily detect diabetes in the same individuals. The concordance between the FPG and 2-h PG test is imperfect, as is the concordance between A1C and either glucose-based test. Compared with FPG and A1C cut points, the 2-h PG value diagnoses more people with prediabetes and diabetes. In people in whom there is discordance between A1C values and glucose values, FPG and 2-h PG are more accurate.

Fasting plasma glucose has been the gold standard diagnostic criterion for T2D and is still the most widely accepted due to its availability, low cost, and compatibility with automated clinical chemistry analyzers. Among the disadvantages of FPG are that it requires at least 8 h fasting, shows substantial biological and diurnal variability, reflects only a single point in time, and the samples involved present stability issues. Despite this, FPG is still widely used individually and as part of blood chemistry panels.

Glycated hemoglobin A1c is directly related to long-term average blood glucose levels and A1C level is strongly correlated with the development of complications due to hyperglycemia. A1C has several advantages compared with FPG and OGTT, including greater convenience (fasting not required), greater preanalytical stability, and less day-to-day perturbations during stress, changes in diet, or illness. However, these advantages may be offset by the lower sensitivity of A1C at the designated cut point, greater cost, and the imperfect correlation between A1C and average glucose in certain individuals. Moreover, age, race, ethnicity, and any clinical condition that alters the lifetime of erythrocytes or hemoglobin levels can alter A1C independent of glucose concentration. A1C testing using the ≥6.5% diagnostic threshold only diagnoses 30% of the total T2D cases identified through A1C, FPG, and OGTT collectively. Also, there is a low correlation between A1C and FPG, insulin resistance, and insulin secretion [Cowie C C, Rust K F, Byrd-Holt D D, Gregg E W, Ford E S, Geiss L S, Bainbridge K E, Fradkin J E. Prevalence of diabetes and high risk for diabetes using AIC criteria in the U.S. population in 1988-2006. Diabetes Care 2010; 33:562-568. doi: 10.2337/dc09-1524].

Oral glucose tolerance test is a marker of early impaired glucose homeostasis and is a more sensitive method of impaired glucose tolerance (IGT) and diabetes diagnosis than FPG and A1C. However, OGTT is relatively costly, can be complicated, and have low reproducibility in some settings. The test protocol requires that the patient ingest an oral load of 75 g of glucose and undergo multiple blood draws over a two-hour period, which can be inconvenient and invasive for the patient. The need for timed samples creates logistical and analytical constraints. Despite its indication for T2D screening by the ADA, OGTT is not usually performed on non-pregnant adults. The OGTT performed between 24 and 28 weeks of gestation is the gold standard for the diagnosis of gestational diabetes (GD), with universal screening advised in populations with a high prevalence of T2D and obesity. In view of the cumbersome nature and poor reproducibility of the OGTT, it is necessary to look for and identify a more robust, convenient, and accurate biomarker for the diagnosis of GD.

So, in summary, there is an unmet clinical need to identify easily measurable and strong biomarkers, which are even more reliable than the traditional OGTT. The present invention is focused on solving this problem and a new and innovative strategy is herein provided for the diagnosis, monitoring or prognosis of IGT/diabetes or prediabetes; or for screening or selecting patient candidates to be subjected to OGTT.

### DESCRIPTION OF THE INVENTION

### Brief Description of the Invention

The present invention refers to an in vitro method for the diagnosis, monitoring or prognosis of IGT/diabetes or prediabetes; or for screening or selecting patient candidates to be subjected to OGTT, which comprises assessing the amount of a protein, selected from the group consisting of: Rbp, Agp, CFH and/or FN, which is/are bound to GAGs. Kindly note that GAGs, also known as mucopolysaccharides, include heparan sulphate (HS), chondroitin sulphate (CS), dermatan sulphate (DS), keratan sulphate (KS), and hyaluronan (HA). Chemically, GAGs are diverse linear carbohydrate chains consisting of repeating units of disaccharides composed of a uronic acid moiety (d-glucuronic or 1-iduronic acid) and an amino sugar (d-galactosamine or d-glucosamine) except for keratan sulphate, which contains repeating units of glucosamine and galactosamine. The amino sugars and uronic acid residues are often sulphated, except in the case of hyaluronan, which impart a high negative charge to the molecules and ensure linearity. Heterogeneity in the GAGs is due to differences in chain length, disaccharide content, disaccharide modifications, degree of sulfation, and charge density. Thus, GAGs are primarily large, heterogeneous, highly charged molecules. GAGs are components of endogenous large heterogeneous molecules known as the proteoglycans (PG). Proteoglycans consist of a protein core with varying numbers of GAGs attached. Proteoglycans are produced by all cells and can be found in the cell membranes, in secretory granules, or released into the surrounding extracellular environment [Saito A, Munakata H. Analysis of plasma proteins that bind to glycosaminoglycans. Biochim Biophys Acta 2007; 1770:241-246. doi: 10.1016/j.bbagen.2006.10.015] [Taylor K R, Gallo R L. Glycosaminoglycans and their proteoglycans: host-associated molecular patterns for initiation and modulation of inflammation. FASEB J. 2006; 20:9-22. doi: 10.1096/fj.05-4682rev].

Particularly, the results provided by the inventors (see Example 2 set below) show a predictive value of the proteins Rbp, Agp, FN and/or CFH bound to GAGs. So, they can be used for the diagnosis, monitoring or prognosis of IGT/diabetes or prediabetes; or for screening or selecting patient candidates to be subjected to OGTT. Interestingly, the results provided herein by the inventors show a predictive value of Rbp, Agp, FN and/or CFH bound to GAGs for inferring blood glucose levels 2 hours after ingestion of 75 g of glucose, i.e., after performing OGTT.

Therefore, according to the present invention, assessing the amount of Rbp, Agp, FN and/or CFH bound to GAGs could be used in different scenarios:
Quick and easy detection of IGT/diabetes and prediabetes.
Prediction tool for the development of IGT/diabetes/prediabetes.
Follow-up and monitoring of patients with IGT/diabetes/prediabetes.
Reduction of the number of OGTT performed by two routes:
   Screening of subjects with normal fasting glucose who may have IGT/diabetes and would be OGTT candidates.
   Not having to repeat OGTT to confirm a diagnosis of IGT/diabetes.
Substitution of OGTT by fasting levels of Rbp, Agp, FN and/or CFH bound to GAGs.

Consequently, the first embodiment of the present invention refers to an in vitro method (hereinafter first method of the invention) for the diagnosis, monitoring or prognosis of IGT/diabetes or prediabetes which comprises assessing the amount of a protein, selected from the group consisting of: Rbp, Agp, FN, and/or CFH, which is/are bound to GAGs in a biological sample obtained from the subject, wherein the determination of a higher amount of a protein, selected from the group consisting of: Rbp, Agp, FN, and/or CFH, bound to GAGs, as compared with a pre-established threshold value, is an indication that the subject may be suffering from IGT/diabetes or/and prediabetes, or that the subject has a poor prognosis.

In a preferred embodiment, the method of the invention is characterized in that it is performed once an OGTT is carried out.

The second embodiment of the invention refers to an in vitro method (hereinafter second method of the invention) for screening or selecting patient candidates to be subjected to OGTT which comprises assessing the amount of a protein, selected from the group consisting of: Rbp, Agp, FN, and/or CFH, which is/are bound to GAGs in a biological sample obtained from the patient, wherein the determination of a higher amount of a protein, selected from the group consisting of: Rbp, Agp, FN, and/or CFH, which is bound to GAGs as compared with a pre-established threshold value, is an indication that the patient may be subjected to OGTT.

In a preferred embodiment, the above methods are performed in combination with the determination of fasting plasma glucose (FPG).

In a preferred embodiment, the biological samples are blood, plasma or serum.

The third embodiment of the present invention refers to the in vitro use of a protein selected from the group consisting of: Rbp, Agp, FN, and/or CFH that is/are bound to GAGs, for the diagnosis, monitoring or prognosis of IGT/diabetes or/and prediabetes; or for screening or selecting patient candidates to be subjected to OGTT.

The fourth embodiment of the present invention refers to a kit consisting of tools and/or reagents for determining the amount of a protein selected from the group consisting of: Rbp, Agp, FN and/or CFH that is/are bound to GAGs.

In a preferred embodiment the kit is an immunoassay which measures the presence or concentration of the above proteins bound to GAGs through the use of an antibody as reagent.

The fifth embodiment of the present invention refers to the use the above defined kit for the diagnosis, monitoring or prognosis of IGT/diabetes or/and prediabetes; or for screening or selecting patient candidates to be subjected to OGTT.

Alternatively, the present invention also refers to:

A method for detecting the amount of a protein selected from the group consisting of: Rbp, Agp, FN, and/or CFH which is/are bound to GAGs in a test sample from a human subject who may be suffering from IGT/diabetes or/and prediabetes, the method comprising the use of an immunoassay to detect and quantify the amount of Rbp, Agp, FN, and/or CFH which is/are bound to GAGs by, for instance:
a) Adding the biological sample to be analysed on the solid support,
b) Adding an antibody which may be conjugated with a detectable labelling agent,
c) Detecting and/or quantifying the signal obtained.

A method for analysing a biological sample prior to diagnosis, monitoring or prognosis of IGT/diabetes or prediabetes; or prior to screening or selecting patient candidates to be subjected to OGTT, comprising analysing the biological sample to assess the amount of Rbp, Agp, FN, and/or CFH which is/are bound to GAGs, wherein the determination of a higher amount of a protein, selected from the group consisting of: Rbp, Agp, FN, and/or CFH, bound to GAGs, as compared with a pre-established threshold value, is an indication that the subject may be suffering from IGT/diabetes or prediabetes, that the subject has a poor prognosis, or that the subject may be subjected to OGTT.

In a preferred embodiment, the present invention is a computer-implemented invention, wherein a processing unit (hardware) and a software are configured to:
Receive the amount of a protein, selected from the group consisting of: Rbp, Agp, FN and/or CFH, bound to GAGs,
Process the amount for finding variations or deviations,
Provide an output through a terminal display of the amount of a protein, selected from the group consisting of: Rbp, Agp, FN and/or CFH, bound to GAGs, wherein the determination of a higher amount of a protein, selected from the group consisting of: Rbp, Agp, FN and/or CFH, bound to GAGs, as compared with a pre-established threshold value, is an indication that the subject may be suffering from IGT/diabetes or prediabetes, that the subject has a poor prognosis, or that the subject may be subjected to OGTT.

The last embodiment of the present invention refers to a method for treating patients suffering from IGT/diabetes or prediabetes, which comprises the administration of a therapeutically effective amount of treatment known in the prior art to treat diabetes, the method comprising performing, as a first step, the above first and or second methods of the invention.

Possible treatments to treat diabetes are depicted below:

| | |
|---|---|
| Class | Compounds |

Noninsulin glucose-lowering agents

| | |
|---|---|
| Biguanides | Metformin |
| Sulfonylureas (2^{nd} generation) | Glimepiride, Glipizide, Glyburide |
| Thiazolidinediones | Pioglitazone, Rosiglitazone |
| α-Glucosidase inhibitors | Acarbose, Miglitol |
| Meglitinides (glinides) | Nateglinide, Repaglinide |
| DPP-4 inhibitors | Alogliptin, Saxagliptin, Linagliptin, Sitagliptin |
| SGLT2 inhibitors | Ertugliflozin, Dapagliflozin, Canagliflozin, Empagliflozin |
| GLP-1 RAS | Exenatide (extended release), Exenatide, Dulaglutide, |
| | Semaglutide, Liraglutide, Lixisenatide |
| Bile acid sequestrant | Colesevelam |
| Dopamine-2 agonist | Bromocriptine |
| Amylin mimetic | Pramlintide |

Insulins

| | |
|---|---|
| Rapid-acting | Lispro follow-on product, Lispro, Lispro-aabc, Glulisine, |
| | Aspart, Aspart (“faster acting product”), Inhaled insulin |
| Short-acting | human regular |
| Intermediate-acting | human NPH |
| Concentrated human regular insulin | U-500 human regular insulin |
| Long-acting | Glargine follow-on products, Glargine, Detemir, |
| | Degludec |
| Premixed insulin products | NPH/regular 70/30, Lispro 50/50, Lispro 75/25, Aspart |
| | 70/30 |
| Premixed insulin/GLP-1 RA products | Glargine/Lixisenatide, Degludec/Liraglutide |

For the purpose of the present invention the following terms are defined:
The term “comprising” means including, but it is not limited to, whatever follows the word “comprising”. Thus, use of the term “comprising” indicates that the listed elements are required or mandatory, but that other elements are optional and may or may not be present.
By “consisting of” means including, and it is limited to, whatever follows the phrase “consisting of”. Thus, the phrase “consisting of” indicates that the listed elements are required or mandatory, and that no other elements may be present.
By “effective dose or amount” is intended an amount that, when administered as described herein, brings about a measurable response in the host. The exact amount required will vary from subject to subject, depending on the age, and general condition of the subject, the severity with which the disease has been/is progressed, mode of administration, and the like. An appropriate “effective” amount in any individual case may be determined by one of ordinary skill in the art using routine experimentation, based upon the information provided herein.
According to the present invention, a reference value can be a “pre-established threshold value” or a “cut-off” value. Typically, a “threshold value” or “cut-off value” can be determined experimentally, empirically, or theoretically. According to the present invention, the “pre-established threshold” value refers to a value previously determined in subjects who are nonsuffering from diabetes or prediabetes. Thus, for instance, the subject is likely to suffer from IGT/diabetes or prediabetes, or to have a poor prognosis, when a higher amount/percentage of a protein, selected from the group consisting of: Rbp, Agp, FN, and/or CFH, bound to GAGs is identified, as compared with a pre-established “threshold value”. A “threshold value” can also be arbitrarily selected based upon the existing experimental and/or clinical conditions, as would be recognized by a person of ordinary skilled in the art. The “threshold value” has to be determined in order to obtain the optimal sensitivity and specificity according to the function of the test and the benefit/risk balance (clinical consequences of false positive and false negative). Typically, the optimal sensitivity and specificity (and so the “threshold value”) can be determined using a Receiver Operating Characteristic (ROC) curve based on experimental data.

### DESCRIPTION OF THE FIGURES

FIG. 1. Relationship of basal serum levels of four protein biomarkers studied with glucose levels at 2-h PG. GAG, glycosaminoglycans; RbpB, basal retinol-binding protein 4; RbpBD, basal retinol-binding protein 4 bound to GAG; RbpBBD, percentage of basal RbpB bound to GAG; AgpB, basal alpha-1-acid glycoprotein; AgpBD, basal alpha-1-acid glycoprotein bound to GAG; AgpBBD, percentage of basal Agp bound to GAG; FNB, basal fibronectin; FNBD, basal fibronectin bound to GAG; FNBBD percentage of basal FN bound to GAG; CFHB, basal complement factor H; CFHBD, basal complement factor H bound to GAG; CFHBBD, percentage of basal CFH bound to GAG; OGTT, serum glucose levels (mg/dL) 2 hours after an oral intake of 75 g of glucose.

FIG. 2. Spearman's correlation between Rbp, Agp, FN and CFH (total and bound to GAGs) at baseline for all subjects and stratified by sex. RbpB, basal retinol-binding protein 4; RbpBD, basal Rbp bound to GAG; RbpBBD, percentage of basal Rbp bound to GAG; AgpB, basal alpha-1-acid glycoprotein; AgpBD, basal Agp bound to GAG; AgpBBD, percentage of basal Agp bound to GAG; FNB, basal fibronectin; FNBD, basal fibronectin bound to GAG; FNBBD percentage of basal FN bound to GAG; CFHB, basal complement factor H; CFHBD, basal CFH bound to GAG; CFHBBD, percentage of basal CFH bound to GAG. * denotes significance at p<0.05; ** denotes significance at p<0.01; *** denotes significance at p<0.001.

### DETAILED DESCRIPTION OF THE INVENTION

The present invention is illustrated by means of the Examples set below without the intention of limiting its scope of protection.

### Example 1. Material and Methods

### Example 1.1. Sample Preparation

Blood samples were drawn in the morning after at least an 8 h overnight fast. Venous blood samples were collected using a serum separator tube (SST). Samples were allowed to clot for 30 minutes at room temperature before centrifugation for 15 minutes at 1000 g. Serum was collected and assayed immediately or was aliquot and store at −80° C. All participants underwent a standard 75 g OGTT with serum collected at 0 (fasting/basal) and 2 h.

### Example 1.2. Laboratory Assays

Glucose was determined in plasma samples by the glucose oxidase peroxidase method on a fully automatic analyzed (ADVIA 2400 from Siemens Healthcare Diagnostics). HbA1c was determined by high-performance liquid chromatography on a Menarini Diagnostics HA-8160 analyzer. Insulin was determined by an immunochemometric assay on a Siemens ADVIA Centaur. All laboratory analyses were performed on the day of sample collection in the Clinical Biochemistry Laboratory of the University Hospital of Santiago de Compostela.

### Example 1.3. Assay Procedure for Separating the Proteins Bound to Glycosaminoglycans

12.2 mg DMB (1.9-dimethylmethylene blue, Serva) was dissolved in 1 ml of ethanol 95%. Then, it was added to 99 ml of 0.2M sodium formiate buffer (formic acid+sodium formiate, pH 3.5). This DMB stock solution is stable in the dark for 30 days at room temperature. DMB stock solution was mixed 1:0.25 with serum (100 μl serum+25 μl DMB solution) and vortex for 10 sec. The mixture was incubated at room temperature during 15 minutes for the formation of a precipitate, and centrifugated at 10.000 g×10 min at 4° C. Carefully, the supernatant was removed by pipetting; and the precipitate containing proteins bound to sulfated GAG was resuspended in PBS (Phosphate Buffered Saline) [Alonso-Sampedro M, Álvarez-González V, Colón-Mejeras C, García-González M, Lamas-González O. Method for separating the fraction bound to glycosaminoglycans and applications thereof. PCT WO 2017/042416 A1].

### Example 1.4. Sample Preparation of the GAG-Bound Proteins for Subsequent Studies (Proteomics and ELISA)

All samples were prepared in duplicate and were resuspended 1× in PBS (100 μl of serum precipitate was resuspended by 100 μl PBS). Its quality was checked by determining the total protein concentration by Bradford assay (Bio-Rad). The coefficient of variation (CV) of the duplicates was calculated, and if it was higher than 30%, the sample was discarded, and the procedure was repeated. The samples of the GAG-bound proteins used in this study had on average a CV of 8.88% (0.21-28.64%). Once the quality was checked, the duplicates were joined in a single tube and stored at −20° C. until use.

### Example 1.5. Proteomic Studies Using SWATH-MS (Sequential Window Acquisition of all Theoretical Mass Spectra)

The combination between SWATH-MS and DIA-MS (Data-independent Acquisition Mass Spectrometry) gives a protein quantification between different sample groups. Therefore, this method combines the targeted data extraction from DIA-MS files from previously identified proteins using higher-resolution mass analysis to perform a more accurate protein quantification.

The more abundant proteins from serum samples were removed adding DTT directly to 30 μl of serum at room temperature followed by removal of the precipitate via centrifugation. The supernatants were transferred to clean tubes. The GAG-bound proteins were used directly. Next, the samples were submitted to an in-gel concentration and in-gel digestion. From each sample, 4 μg of digested peptides was injected in a reversed-phase LC separation using the Nano-LC Ultra system Eksigent 425 in micro mode, coupled with the ekspert 400 autosampler (ABSciex). The eluted peptides are analyzed on a High-Resolution Quadrupole Time-of-Flight (QTOF instrument), TripleTOF 6600+ system (ABSciex). The pooled sample from each group was analyzed in the IDA mode to generate the spectral library. A DDA/IDA method was created using Analyst TF 1.7.1 with one survey scan and 60 candidate ion scans per cycle. The extraction of all the precursor information from these runs was done using software called Peak View that generates the entries needed for the Variable Window Calculator. With this tool, all the Q1 transmission windows needed were generated. The windows generated are exported to a text file that can finally be integrated into the Acquisition Control software (ABSciex). SWATH-MS data analysis was performed using Peak View 2.2 and SWATH-MS Microapp 2.0 (ABSciex). A detailed protocol can be found at Chantada-Vazquez et al, 2021. [Chantada-Vázquez M P, García Vence M, Serna A, Núñez C, Bravo S B. (2021). SWATH-MS Protocols in Human Diseases. Methods in Molecular Biology (Vol. 2259, pp. 105-141). Humana Press Inc. https://doi.org/10.1007/978-1-0716-1178-4_7]

### Example 1.6. Determination of Retinol Binding Protein 4 Levels

Levels of Rbp were determined using the #ab196264 Human RBP4 SimpleStep ELISA Kit from abcam. The lowest limit of detection was 2.6 pg/ml. The mean intra- and interassay CV were 5.1% and 8.9%, respectively. The assay was performed following the instructions of the manufacturer. Serum samples were diluted sequentially (1:50, 1:40, 1:40, and 1:5) using Sample Diluent NS (final dilution 1:400000). The GAG-bound proteins were diluted sequentially (1:50 and 1:40) using Sample Diluent NS (final dilution 1:2000). To perform the assay, samples or standards were added to the wells, followed by the antibody mix. After 1 h of incubation at room t^{a}, the wells were washed to remove unbound material. TMB substrate was added and during incubation is catalyzed by HRP, generating blue coloration. This reaction was then stopped by addition of Stop Solution completing any color change from blue to yellow. Signal was generated proportionally to the amount of bound analyte and the intensity was measured at 450 nm. The concentration of Rbp was determined by interpolating blank control subtracted absorbance values against the standard curve. The resulting value was multiplied by the dilution factor to obtain the concentration in the sample. The measurement results in pg/ml were converted to μg/ml.

### Example 1.7. Determination of Fibronectin Levels

Levels of FN were determined using the #ab219046 Human Fibronectin SimpleStep ELISA Kit from abcam. The lowest limit of detection was 20.6 μg/ml. The mean intra- and interassay CV were 5.0% and 8.3%, respectively. The assay was performed following the instructions of the manufacturer. Serum samples were diluted sequentially (1:50, 1:40 and 1:20) using Sample Diluent NS (final dilution 1:40000). The GAG-bound proteins were diluted sequentially (1:50, 1:40 and 1:10) using Sample Diluent NS (final dilution 1:20000). To perform the assay, samples or standards were added to the wells, followed by the antibody mix. After 1 h of incubation at room t^{a}, the wells were washed to remove unbound material. TMB substrate was added and during incubation is catalyzed by HRP, generating blue coloration. This reaction was then stopped by addition of Stop Solution completing any color change from blue to yellow. Signal was generated proportionally to the amount of bound analyte and the intensity was measured at 450 nm. The concentration of FN was determined by interpolating blank control subtracted absorbance values against the standard curve. The resulting value was multiplied by the dilution factor to obtain the concentration in the sample. The measurement results in pg/ml were converted to μg/ml.

### Example 1.8. Determination of Alpha 1 Acid Glycoprotein Levels

Levels of Agp were determined using the #ab243675 Human alpha 1 Acid Glycoprotein SimpleStep ELISA Kit from abcam. The lowest limit of detection was 68.5 ng/ml. The mean intra- and interassay CV were 5.9% and 5.4%, respectively. The assay was performed following the instructions of the manufacturer. Serum samples were diluted sequentially (1:50 and 1:40) using 1× Wash Buffer PT, and then were further diluted sequentially (1:40 and 1:2) using Sample Diluent NS (final dilution 1:160000). The GAG-bound proteins were diluted 1:50 using 1× Wash Buffer PT, and then diluted 1:40 using Sample Diluent NS (final dilution 1:2000). To perform the assay, samples or standards were added to the wells, followed by the antibody mix. After 1 h of incubation at room t^{a}, the wells were washed to remove unbound material. TMB substrate was added and during incubation is catalyzed by HRP, generating blue coloration. This reaction was then stopped by addition of Stop Solution completing any color change from blue to yellow. Signal was generated proportionally to the amount of bound analyte and the intensity was measured at 450 nm. The concentration of Agp was determined by interpolating blank control subtracted absorbance values against the standard curve. The resulting value was multiplied by the dilution factor to obtain the concentration in the sample. The measurement results in ng/ml were converted to μg/ml.

### Example 1.9. Determination of Complement Factor H Levels

Levels of CFH were determined using the #ab252359 Human Complement factor H SimpleStep ELISA Kit from abcam. The lowest limit of detection was 74 μg/ml. The mean intra- and interassay CV were 2.5% and 5.8%, respectively. The assay was performed following the instructions of the manufacturer. Serum samples were diluted sequentially (1:100 and 1:100) using 1× Wash Buffer PT, and then were further diluted sequentially 1:20 using Sample Diluent NS (final dilution 1:200000). The GAG-bound proteins were diluted 1:100 using 1× Wash Buffer PT, and then diluted 1:5 using Sample Diluent NS (final dilution 1:500). To perform the assay, samples or standards were added to the wells, followed by the antibody mix. After 1 h of incubation at room t^{a}, the wells were washed to remove unbound material. TMB substrate was added and during incubation is catalyzed by HRP, generating blue coloration. This reaction was then stopped by addition of Stop Solution completing any color change from blue to yellow. Signal was generated proportionally to the amount of bound analyte and the intensity was measured at 450 nm. The concentration of CFH was determined by interpolating blank control subtracted absorbance values against the standard curve. The resulting value was multiplied by the dilution factor to obtain the concentration in the sample. The measurement results in pg/ml were converted to μg/ml.

### Example 1.10. Glycosaminoglycan-Protein Binding Assays

Chondroitin sulfate (CS) sodium salt from shark cartilage [Sigma C4384], Heparan sulfate (HS) sodium salt bovine kidney [Sigma H7640], and Hyaluronic acid (HA) sodium salt from bovine vitreous humor [Sigma H7630] were dissolved in PBS at a concentration of 1 μg/μl. A pool of human sera was incubated in triplicate with PBS or with increasing amounts of CS, HS or AH for one hour at 37° C. GAG-bound proteins were then precipitated and the concentration of GAG-bound Agp or Rbp was determined using the ELISA assays described above. The percentages of Agp or Rbp bound to GAGs were calculated relative to serum without added GAGs (100%) to compare the affinity of both proteins for the different GAGs tested.

### Example 1.11. Statistical Analyses

A descriptive analysis of the sample was conducted to obtain the mean and standard deviation (SD) or the median and range for the quantitative variables. For the correlation studies, Spearman's test was used. To observe the differences between groups the Mann-Whitney test was used.

Logistic regression models were built to predict the probability of being a person with IGT/T2D or prediabetes. Models were performed first entering only FPG or the basal levels of one of the biomarkers (total and bound to GAGs), and then, the combination of FPG and each of the biomarkers. Receiver operating curves were constructed to evaluate the discrimination power of the model and areas under the curve (AUC) were calculated.

Generalized additive models (GAM) with glucose levels after OGTT as the response variable were also performed to verify association with the biomarkers after adjusting by FPG and/or age.

All statistical analyses were carried out in R using the “rms”, “mgcv” and “base” packages. These packages are freely available at cran.r-project.org.

### Example 1.12. Study Population

The A Estrada Glycation and Inflammation Study (AEGIS I; trial NCT01796184 at www.clinicaltrials.gov) is an epidemiological study aimed to evaluate the relationship between inflammation and glycation factors and metabolic diseases, specifically diabetes and cardiovascular disease. Briefly, an age-stratified random sample of the population aged 18 years and older was drawn from Spain's National Health System Registry. There were 1516 subjects agreed to participate in the study (overall participation rate, 68%), made up of 678 men (45%) and 838 women (55%). From November 2012 through March 2015, all subjects were successively convened for one day at the Primary Care Center for evaluation, which comprised (1) an interviewer-administered structured questionnaire that included demographic and anthropometric data; (2) a lifestyle description, including diet, physical exercise, alcohol consumption, and smoking; and (3) fasting venous blood sampling and first void urine. In addition, 622 subjects consented to undergo a 6-day period of continuous glucose monitoring procedures. [Gude F, Diaz-Vidal P, Rúa-Pérez C, Alonso-Sampedro M, Fernández-Merino C, Rey-García J, Cadarso-Suárez C, Pazos-Couselo M, García-López J M, González-Quintela A. Glycemic Variability and Its Association with Demographics and Lifestyles in a General Adult Population. J Diabetes Sci Technol. 2017; 11:780-790. doi: 10.1177/1932296816682031].

The 5-years follow-up study (AEGIS II, PI16/01395) was ended in June 2020. The aim was to assess the efficacy of continuous glucose monitoring in predicting risk of type 2 diabetes, and to develop and validate a prediction risk model for type 2 diabetes after 5-year follow-up. The study comprises all the 1516 participants from the AEGIS I. After written informed consent, all the participants were re-evaluated for one day in the Primary Care Center. The standard workup includes a structured questionnaire, and clinical examination that allows for categorization of participants in terms of diabetes status according to the diagnostic criteria of the American Diabetes Association. Seven hundred and forty-six individuals agreed to participate (overall participation rate, 66.5%), 327 men (43.8%) and 419 women (56.2%). In addition, 236 subjects agreed to repeat the continuous glucose monitoring. The events of the incidence of diabetes and chronic diseases frequent in the population were monitored.

It was performed an OGTT on individuals not previously diagnosed with diabetes. The OGTT was used to screen IGT/T2D and to provide a critical understanding of its future evolution. We enrolled 299 subjects, without prior diagnosis of diabetes, to undergo a 75 g OGTT. According to the diagnostic criteria of the ADA (Table 1), the subjects were assigned to one of the three groups: i) normal glucose tolerance (NGT); ii) impaired glucose tolerance (IGT); iii) T2D (Table 2).

The 299 subjects who performed the OGTT were also classified according to their basal glycemic state following the ADA criteria (Table 1) into three groups: i) normoglycemia; ii) prediabetes; iii) T2D (Table 3).

TABLE 2
Classification according to the glycemic state at 2h-PG of the
individuals who performed the OGTT.

| | Women | Men | All |
|---|---|---|---|
| NGT | 166 (91.7%) | 97 (82.2%) | 263 (87.9%) |
| IGT | 12 (6.6%) | 16 (13.6%) | 28 (9.4%) |
| T2D | 3 (1.7%) | 5 (4.2%) | 8 (2.7%) |
| Total | 181 (60.5%) | 118 (39.5%) | 299 (100%) |

NGT, normal glucose tolerance;
IGT, impaired glucose tolerance;
T2D, type 2 diabetes

TABLE 3
Classification according to the basal glycemic state of the
individuals who performed the OGTT.

| | Women | Men | All |
|---|---|---|---|
| Normoglycemia | 125 (69.1%) | 88 (74.6%) | 213 (71.2%) |
| Prediabetes | 54 (29.8%) | 30 (25.4%) | 84 (28.1%) |
| T2D | 2 (1.1%) | 0 (0.0%) | 2 (0.7%) |
| Total | 181 (60.5%) | 118 (39.5%) | 299 (100%) |

T2D, type 2 diabetes

In both studies (AEGIS II and I), remaining blood and urine collected was freeze and conformed an ISCIII-collection (C.0004087).

### Example 2. Results

### Example 2.1. Proteomic Pilot-Study

Proteomics has been widely used in basic research and clinical diagnosis of human diseases; involves analysis, characterization, and classification of all protein in a sample. In particular, differential proteomics is a powerful technique based on comparing changes in protein levels between samples, which allows both to investigate the disease as a whole and to search for protein markers.

We conducted a proteomic pilot-study using the quantitative methodology SWATH-MS (Sequential Window Acquisition of all Theorical Mass Spectra). Xunta de Galicia (GPC-GAIN, IN607B 2018/1) funded the study. Of the 299 subjects who underwent the OGTT (Table 2), we selected 15 men (60-70 years old): nine with NGT, four with IGT and two with T2D. In the serum samples analyzed, we were able to measure the expression levels of 273 proteins. Table 4 shows the differentially expressed proteins (p-value<0.05 and >1.5-fold change up or down) between individuals classified as NGT (n=9) versus IGT/T2D (n=6).

TABLE 4
Potential proteins as biomarkers in diabetes from SWATH-MS
proteomic analysis

| UniProt # | Protein | Gene |
|---|---|---|
| P02753 | Retinol-binding protein 4 | RBP4 |
| P02763 | Alpha-1-acid glycoprotein 1 | ORM1/AGP1 |
| P02751 | Fibronectin | FN1 |
| P08603 | Complement factor H | CFH |
| P10909 | Clusterin | CLU (APOJ, CLI, |
| | | KUB1) |
| P01701 | Immunoglobulin lambda variable 1-51 | IGLV1-51 |
| P18428 | Lipopolysaccharide-binding protein | LBP |
| P02765 | Alpha-2-HS-glycoprotein | AHSG (FETUA) |
| P00739 | Haptoglobin-related protein | HPR |
| P07737 | Profilin-1 | PFN1 |
| P00746 | Complement factor D | CFD (DF, PFD) |
| P24821 | Tenascin | TNC (HXB) |
| P35908 | Keratin, type II cytoskeletal 2 epidermal | KRT2 (KRT2A, |
| | | KRT2E) |
| P01024 | Complement C3 | C3 (CPAMD1) |
| P01704 | Immunoglobulin lambda variable 2-14 | IGLV2-14 |
| P05546 | Heparin cofactor 2 | SERPIND1 (HCF2) |
| O14791 | Apolipoprotein L1 | APOL1 (APOL) |
| Q96KN2 | Beta-Ala-His dipeptidase | CNDP1 (CN1, |
| | | CPGL2) |
| Q13093 | Platelet-activating factor acetylhydrolase | PLA2G7 (PAFAH) |
| P36955 | Pigment epithelium-derived factor | SERPINF1 (PEDF) |

### Example 2.2. Selection of the Biomarkers to Validate by ELISA

From the results of the proteomic pilot-study, we selected the most promising differentially expressed potential protein biomarkers (total or bound to glycosaminoglycans) with commercial ELISA available (funded by GRC-GAIN, IN607A 2021/1, Xunta de Galicia). The four selected proteins were retinol-binding protein 4 (Rbp), alpha-1-acid glycoprotein (Agp/ORM), fibronectin (FN) and complement factor H (CFH).

Retinol-binding protein 4 is a member of the lipocalin family and the major transport protein of the hydrophobic molecule retinol, also known as vitamin A, in the circulation. Retinol binds to Rbp in the hepatocyte and after associating with transthyretin (TTR), the retinol/Rbp/TTR complex is released into the bloodstream and delivers retinol to tissues via binding to specific membrane receptors. Rbp is implicated in a variety of human conditions that include impaired vision and ocular diseases, disorders of glucose and lipid homeostasis, and cardiovascular diseases. [Steinhoff J S, Lass A, Schupp M. Biological functions of RBP4 and its relevance for human disease. Front Physiol 2021; 12:659977. doi: 10.3389/fphys.2021.659977. eCollection 2021].

Alpha-1-acid glycoprotein (also known as orosomucoid) is an abundant immunomodulatory protein in circulation, which is induced by stressful conditions such as infections and inflammation, and elevated levels have been found in diabetic patients. Expression of Agp has been linked with metabolic signaling, including hyperglycemia, and has been suggested to modulate immune responses to protect adipose tissue from inflammation and metabolic dysfunction. [Wurtz P, Tiainen M, Makinen V P, Kangas A J, Soininen P, Saltevo J, Keinanen-Kiukaanniemi S, Mantyselka P, Lehtimaki T, Laakso M, Jula A, Kahonen M, Vanhala M, Ala-Korpela M. Circulating metabolite predictors of glycemia in middle-aged men and women. Diabetes Care 2012; 35:1749-1756. doi: 10.2337/dc11-1838].

Fibronectin is a large dimeric glycoprotein that is expressed in various cell and tissue types and participates in multiple functions such as cell adhesion, growth, migration and differentiation. Cellular form of FN is synthetized by endothelial cells, fibroblasts and smooth muscle cells. FN is one of the most reliable proteins that can be estimated as a plasma indicator protein for endothelial function and related pathological disorders. Significant elevation of circulating FN has been reported in various metabolic syndromes associated with endothelial function, such as diabetes. The change in the levels of serum or plasma cellular FN may reflect the extent of matrix changes and vessel wall damage in patients with diabetes. Although various forms of FN have been implicated to have a role in metabolic disorders like diabetes the specific glycosylated version in question has only been investigated in just few studies. All forms of FN are glycosylated but, in these studies, the term glycosylated refers to specific sialyated glucose that SNA lectin recognizes. [Alanen J, Appelblom H, Korpimaki T, Kouru H, Sairanen M, Gissler M, Ryynanen M, Nevalainen J. Glycosilated fibronectin as a first trimester marker for gestational diabetes. Arch Gynecol Obstet 2020, 302:853-860. doi: 10.1007/s00404-020-05670-8].

Complement factor H is a relatively abundant glycoprotein that ensures downregulation of the complement alternative pathway, a branch of innate immunity, upon interaction with specific glycans on host cell surfaces. CFH recognizes and binds to self-surfaces via sialic acid and GAGs. CFH contains multiple GAG-binding sites that have the capacity to cooperate or interfere with one another under physiological circumstances. The GAG-binding sites can modulate the ability to distinguish between GAGs according to type and density of sulfation. [Herbert A P, Deakin J A, Schmidt C Q, et al. Structure shows that a glycosaminoglycan and protein recognition site in factor H is perturbed by age-related macular degeneration-linked single nucleotide polymorphism. J Biol Chem. 2007; 282 (26): 18960-18968. doi: 10.1074/jbc.M609636200].

### Example 2.3. Validation of the Biomarkers by ELISA

All four biomarkers could not be analyzed in the 299 subjects who underwent the OGTT (Table 2). Because the number of individuals with values in the type 2 diabetes range is low (n=8), only the combined IGT/T2D (2-h PG>139 mg/dL) was studied. Analyses were always performed on all subjects and stratified by sex.

The clinical characteristics of the 280 individuals analyzed according to their glycemic status at 2-h PG (NGT and IGT/T2D) are shown in Table 5. As expected, as the glycemic status worsens (from NGT to IGT/T2D), there was a significant increase in BMI, FPG, A1C, fructosamine and insulin. No differences in age were found in relation to glycemic status. In general, no differences were found between men and woman.

Table 6 shows the basal serum levels of the four selected biomarkers (total and GAG-bound) in individuals classified as NGT and IGT/T2D in all subjects and stratified by sex. The levels of Rbp increase (35.03 μg/ml vs 39.36 μg/ml) without reaching statistical significance, as moved from NGT to IGT/T2D (p=0.054) for all subjects. No differences were observed in the levels of Rbp if we analyzed men and women separately. However, Rbp levels were lower in women than in men (31.31 μg/ml vs 38.91 μg/ml) in the NGT group (p<0.001). The levels of Rbp/GAG-bound increase significantly as moved from NGT to IGT/T2D in all subjects and in men and women separately (0.70 vs 1.00 μg/ml, 0.82 vs 1.18 μg/ml and 0.65 vs 0.85 μg/ml, respectively). Again, the Rbp/GAG-bound levels were lower in women than in men (0.65 vs 0.82 μg/ml, respectively) in the NGT group (p<0.001). The levels of % Rbp/GAG-bound increase significantly as moved from NGT to IGT/T2D (0.70% vs 1.00%) in all subjects (p=0.004). No differences were observed in the levels of total Agp. The levels of Agp/GAG-bound (6.17 vs 6.33 μg/ml) and the % Agp bound to GAGs (0.78% vs 0.90%) were increase significantly in the subjects with IGT/T2D (p<0.05). No differences were observed in the levels of FN, FN/GAG-bound or % FN/GAG-bound between NGT and IGT/T2D subjects. In the levels of CFH, a significant increase (50.23 vs 61.79 μg/ml) was observed as moved from a state of NGT to IGT/diabetes in all subjects (p=0.012) and in women (49.22 vs 52.24 μg/ml, p=0.007). The levels of CFH/GAG-bound increase significantly as moved from NGT to IGT/T2D in all subjects and in men and women separately (0.55 vs 0.62 μg/ml, 0.55 vs 0.62 μg/ml and 0.56 vs 0.74 μg/ml, respectively). No differences were observed in the levels of % CFH bound to GAG in any of the groups tested.

In summary, both Rbp/GAG-bound and CFH/GAG-bound levels showed a significant increase in IGT/T2D individuals analyzed both together and separated by sex. The levels of % Rbp/GAG-bound, Agp/GAG-bound and % Agp/GAG-bound showed a significant increase in IGT/T2D individuals in analysis as a whole of men and women losing significance when analyzed separately. The CFH levels showed a significant increase in IGT/T2D individuals (all subjects) and in women.

Our data show an increase in Rbp in individuals with IGT/T2D that is at the limit of statistical significance (p=0.054). However, in the study by Cho et al. found that the Rbp concentration increased significantly from the NGT group to the IGT and T2D groups (18.1, 18.9 and 20.9 μg/ml, respectively, p=0.002). They did not find significant differences between the IGT and NGT groups. Likewise, they found that the serum concentration of Rbp is associated with sex, men having a higher concentration than women (NGT 19.4 vs 16.2; IGT 22.5 vs 18.0; and DM 23.8 vs 20.1 μg/ml, p=0.049). In our study we also observed a significant increase in Rbp and Rbp/GAG-bound in men compared to women but only in the NGT group (38.91 vs 31.31 μg/ml; 0.82 vs 0.65%; p<0.001). However, we did not find differences between sexes in the % Rbp bound to GAGs (p>0.05). One possible cause of the discordant results is the different assays used in the measurement of Rbp. Graham et al. measured Rbp levels in subjects with insulin resistance and glucose intolerance and insulin-sensitive subjects with NGT using three commercial assays and a quantitative Western blot assay. They found substantial inconsistency between results with enzyme immunoassays that underestimated the Rbp levels and concluded that Western blot is the most reliable method to measure Rbp. However, we cannot rule out the possibility that this discrepancy was caused by the different measuring systems used (i.e., band intensity by Western blotting vs ELISA optical density) or by the different affinities of the antibodies used. [Cho Y M, Young B S, Lee H, Lee N, Min S S, Kwak S H, Lee H K, Park K S. Plasma retinol-binding protein-4 concentrations are elevated in human subjects with impaired glucose tolerance and type 2 diabetes. Diabetes Care 2006, 29:2457-2461. doi: 10.2337/dc06-0360]. [Graham T E, Wason C J, Blüher M, Kahn B B. Shortcomings in methodology complicate measurements of serum retinol binding protein (RBP4) in insulin-resistant human subjects. Diabetologia 2007, 50:814-823. doi: 10.1007/s00125-006-0557-0].

TABLE 5
Clinical characteristics of the individuals analyzed according to
the glycemic state at 2h-PG for all subjects and stratified by sex.

| | | NGT | IGT/T2D | p-value |
|---|---|---|---|---|
| Age, years | Men | 55.03 ± 14.89 | 59.29 ± 12.29 | 0.555 |
| | Women | 56.58 ± 15.04 | 58.20 ± 15.88 | 0.270 |
| | All | 55.96 ± 14.97 | 58.83 ± 13.69 | 0.243 |
| BMI, kg/m² | Men | 28.72 ± 4.00 | 31.75 ± 5.36 | 0.017 |
| | Women | 28.18 ± 5.13 | 31.79 ± 6.42 | 0.027 |
| | All | 28.40 ± 4.71 | 31.77 ± 5.74 | <0.001 |
| FPG, mg/dL | Men | 86.16 ± 9.15 | 99.67 ± 12.69 | <0.001 |
| | Women | 84.33 ± 10.36 | 97.73 ± 9.04 | <0.001 |
| | All | 85.06 ± 9.92 | 98.86 ± 11.21 | <0.001 |
| A1C, % | Men | 5.39 ± 0.30 | 5.64 ± 0.38 | <0.001 |
| | Women | 5.41 ± 0.37 | 5.89 ± 0.55 | 0.002 |
| | All | 5.41 ± 0.34 | 5.74 ± 0.47 | <0.001 |
| Fructosamine, | Men | 214.85 ± 30.18 | 223.76 ± 29.01 | 0.025 |
| μmol/L | Women | 224.29 ± 31.63* | 254.27 ± 51.59 | 0.237 |
| | All | 220.54 ± 31.34 | 236.47 ± 42.16 | 0.047 |
| Insulin, mUI/L | Men | 8.93 ± 6.15 | 17.20 ± 13.07 | 0.010 |
| | Women | 9.04 ± 4.73 | 11.71 ± 4.14 | 0.001 |
| | All | 8.99 ± 5.34 | 14.91 ± 10.58 | <0.001 |

NGT, normal glucose tolerance;
IGT, impaired glucose tolerance;
T2D, type 2 diabetes;
FPG, fasting plasma glucose;
BMI, body mass index;
A1C, glycosilated hemoglobin A1c.
Data are expressed as mean ± SD.
The p-values were performed using Mann-Whitney test.
p-value: *< 0.05 men vs women. All subjects n = 280 [NGT n = 244; IGT/T2D n = 36]; Women n = 162 [NGT n = 147; IGT/T2D n = 15]; Men n = 118 [NGT n = 97; IGT/T2D n = 21].

Hence, Rbp's causal role in inducing insulin resistance and T2D is still vividly debated within the field. The underlying reasons and whether or not Rbp is indeed actively contributing to insulin resistance is still under investigation. Are there yet unidentified post-translational modifications of Rbp that could mediate this? Our results show that the deterioration of glycemic status could be related to the binding of GAG to Rbp.

Glycosylated plasma FN has been indicated as an early predictor for gestational diabetes, however the studies have been contradictory. Rasanen et al. found in their study a significant difference in glycosylated fibronectin concentrations between the GD group (132±36 mg/L, n=90) and the control group (80±4.0 mg/L, n=92, p<0.001). However, Alanen et al. did not found difference in maternal serum glycosylated FN concentrations between women with consequent GD [447.5 μg/ml, interquartile range (IQR) 254.4-540.9 μg/ml, n=19] and control women (437.6 μg/ml, IQR 357.1-569.1 μg/ml, n=59). Maternal serum FN levels were significantly lower in GD group (224.2 μg/ml, IQR 156.8-270.6 μg/ml), compared to the control group (264.8 μg/ml, IQR 224.6-330.6 μg/ml, p<0.01). There is an ongoing prospective study using maternal serum glycosylated FN and/or OGTT 75 g at 12-15 gestational weeks as a predictor of subsequent GD. In our study, no differences were observed in the levels of FN or in that bound to GAG as moved from a state of normal glucose tolerant to IGT/T2D (p>0.05). [Rasanen J P, Snyder C K, Rao P V, Mihalache R, Heinonen S, Gravett M G, Roberts C T, Nagalla S R. Glycosylated fibronectin as a first-trimester biomarker for prediction of gestational diabetes. Obstet Gynecol 2013, 122:586-94. doi: 10.1097/AOG.0b013e3182a0c88b]. [Alanen J, Appelblom H, Korpimaki T, Kouru H, Sairanen M, Gissler M, Ryynanen M, Nevalainen J. Glycosilated fibronectin as a first trimester marker for gestational diabetes. Arch Gynecol Obstet 2020, 302:853-860. doi: 10.1007/s00404-020-05670-8]. [Huhn E A, Fischer T, Göbl CS, Bernasconi M T, Kreft M, Kunze M, Schoetzau A, Dolzlmuller E, Eppel W, Husslein P, Ochsenbein-Koelble N, Zimmermann R, Baz E, Prompeler H, Bruder E, Hahn S, Hoesli I. Screening of gestational diabetes mellitus in early pregnancy by oral glucose tolerance test and glycosylated fibronectin: study protocol for an international, prospective, multicentre cohort trial. BMJ Open 2016; 6: e012115. doi: 10.1136/bmjopen-2016-012115].

Low-grade, systemic inflammation has been associated with the risk of diabetes. Prospective studies have demonstrated that higher Agp is associated with an increased risk of T2D. In the study of Wurtz et al., Agp was prospectively associated with both fasting and postchallenge glucose. The results suggest that Agp is not only related to metabolic dysfunction cross-sectionally but also as a predictor for future glycemia, and thus highlight the role of prolonged inflammation as a risk marker for attenuating glucose tolerance. Tsuboi et al. have recently demonstrated that serum Agp was related to postload glucose and glucose excursion during OGTT in non-obese young Japanese women. In our study, no differences were observed in the levels of total Agp, but Agp/GAG-bound and % Agp/GAG-bound showed a significant increase in IGT/T2D individuals in analysis as a whole of men and women losing significance when analyzed separately. [Wurtz P, Tiainen M, Makinen V P, Kangas A J, Soininen P, Saltevo J, Keinanen-Kiukaanniemi S, Mantyselka P, Lehtimaki T, Laakso M, Jula A, Kahonen M, Vanhala M, Ala-Korpela M. Circulating metabolite predictors of glycemia in middle-aged men and women. Diabetes Care 2012; 35:1749-1756. doi: 10.2337/dc11-1838]. [Tsuboi A, Minato S, Yano M, Takeuchi M, Kitaoka K, Kurata M, Yoshino G, Wu B, Kazumi T, Fukuo K. Association of serum orosomucoid with 30-min plasma glucose and glucose excursion during oral glucose tolerance tests in non-obese young Japanese women. BMJ Open Diabetes Res Care 2018; 6: e000508. doi: 10.1136/bmjdrc-2018-000508. eCollection 2018].

It has been suggested that CFH was associated with obesity and metabolic disorders. Moreno-Navarrete et al. found that the CFH level significantly increased in patients with altered glucose tolerance and T2D (NGT 175. vs IGT/T2D 195.4 μg/ml), and plasma CFH levels were negatively associated with insulin sensitivity. Li et al. studied whether CFH played a role in gestational diabetes. CFH was higher in GD group than in non-GD controls (280.02 vs 264.20 μg/ml, p=0.014). However, following binary logistic regression, CFH was not independently associated with GD. The CFH levels in 11-17th weeks of gestation may be affected by many factors, including BMI and age. Similarly, our data showed a significant increase in CFH levels in women with IGT/T2D compared to NGT (52.24 vs 49.22 μg/ml, p=0.007), and the same trend was observed in the analysis of both sexes (61.79 vs 50.23 μg/ml, p=0.012). [Moreno-Navarrete J M, Martinez-Barricarte R, Catalán V, et al. Complement factor H is expressed in adipose tissue in association with insulin resistance. Diabetes. 2010; 59 (1): 200-209. doi: 10.2337/db09-0700]. [Li J, Shen Y, Tian H, et al. The role of complement factor H in gestational diabetes mellitus and pregnancy. BMC Pregnancy Childbirth. 2021; 21 (1): 562. Published 2021 Aug. 17. doi: 10.1186/s12884-021-04031-w].

TABLE 6
Basal serum levels of the four biomarkers (total and GAG-bound) according to the
glycemic state at 2h-PG for all subjects and stratified by sex.

| | | NGT | IGT/T2D | p-value |
|---|---|---|---|---|
| Rbp | Men | 38.91 [14.64-109.96] | 41.45 [11.52-109.21] | 0.258 |
| (μg/ml) | Women | 31.31 [9.89-272.15]*** | 34.29 [13.31-69.14] | 0.295 |
| | All | 35.03 [9.89-272.15] | 39.36 [11.52-109.21] | 0.054 |
| Rbp/GAG-bound | Men | 0.82 [0.31-1.95] | 1.18 [0.20-3.04] | 0.011 |
| (μg/ml) | Women | 0.65 [0.20-2.08] ** | 0.85 [0.29-2.36] | 0.004 |
| | All | 0.70 [0.20-2.08] | 1.00 [0.20-3.04] | <0.001 |
| Rbp GAG-bound | Men | 2.06 [0.70-6.15] | 2.67 [1.71-4.54] | 0.095 |
| (%) | Women | 2.09 [0.43-6.85] | 2.21 [1.32-7.23] | 0.078 |
| | All | 2.08 [0.43-6.85] | 2.58 [1.32-7.23] | 0.004 |
| Agp | Men | 822.78 [315.42-2056.69] | 1006.29 [423.96-4230.73] | 0.633 |
| (μg/ml) | Women | 808.98 [393.06-2082.00] | 842.24 [314.47-1683.27] | 0.996 |
| | All | 809.49 [315.42-2082.00] | 900.70 [314.47-4230.73] | 0.624 |
| Agp/GAG-bound | Men | 6.53 [2.32-20.33] | 6.17 [3.16-45.45] | 0.230 |
| (μg/ml) | Women | 6.10 [1.64-36.78] | 6.33 [3.57-29.49] | 0.127 |
| | All | 6.17 [1.64-36.78] | 6.33 [3.16-45.45] | 0.040 |
| Agp/GAG-bound | Men | 0.76 [0.36-2.26] | 0.89 [0.55-3.55] | 0.245 |
| (%) | Women | 0.79 [0.28-4.55] | 0.93 [0.31-2.50] | 0.071 |
| | All | 0.78 [0.28-4.55] | 0.90 [0.31-3.55] | 0.045 |
| FN | Men | 190.12 [110.05-351.23] | 210.99 [157.43-401.10] | 0.653 |
| (μg/ml) | Women | 169.26 [97.45-349.20]* | 165.77 [68.53-238.50]** | 0.069 |
| | All | 177.74 [97.45-351.23] | 187.90 [68.53-401.10] | 0.124 |
| FN/GAG-bound | Men | 84.09 [18.76-161.71] | 103.07 [42.30-177.31] | 0.960 |
| (μg/ml) | Women | 74.92 [21.63-176.90]*** | 74.75 [2.41-144.92] | 0.141 |
| | All | 78.58 [18.76-176.90] | 79.66 [12.41-177.31] | 0.143 |
| FN/GAG-bound | Men | 43.05 [14.10-64.36] | 45.41 [23.25-65.02] | 0.416 |
| (%) | Women | 41.51 [15.15-70.97] | 45.10 [10.78-68.03] | 0.977 |
| | All | 42.69 [14.10-70.97] | 45.25 [10.78-68.03] | 0.455 |
| CFH | Men | 50.23 [17.11-2268.38] | 62.29 [28.14-125.89] | 0.472 |
| (μg/ml) | Women | 49.22 [14.11-222.58] | 52.24 [31.15-96.57] | 0.007 |
| | All | 50.23 [14.11-2268.38] | 61.79 [28.14-125.89] | 0.012 |
| CFH/GAG-bound | Men | 0.55 [0.30-2.33] | 0.62 [0.44-1.66] | 0.020 |
| (μg/ml) | Women | 0.56 [0.25-1.62] | 0.74 [0.40-1.03] | 0.020 |
| | All | 0.55 [0.25-2.33] | 0.62 [0.40-1.66] | <0.001 |
| CFH/GAG-bound | Men | 1.08 [0.03-4.21] | 1.16 [0.54-2.01] | 0.267 |
| (%) | Women | 1.15 [0.24-4.14] | 1.26 [0.77-2.59] | 0.907 |
| | All | 1.14 [0.03-4.21] | 1.18 [0.54-2.59] | 0.441 |

NGT, normal glucose tolerance;
IGT, impaired glucose tolerance;
T2D, type 2 diabetes;
Rbp, retinol-binding protein 4;
Agp, alpha-1-acid glycoprotein;
FN, fibronectin;
CFH, complement factor H;
GAG, glycosaminoglycans.
Data are expressed as median [min-max].
p-value: *< 0.05, **< 0.01, ***< 0.001 men vs women.
The p-values were performed using Mann-Whitney test.
Rbp and Agp: All subjects n = 239 [NGT n = 203; IGT/T2D n = 36]; Women n = 147 [NGT n = 132; IGT/T2D n = 15]; Men n = 92[NGT n = 71; IGT/T2D n = 21]
FN: All subjects n = 199 [NGT n = 163; IGT/T2D n = 36]; Women n = 107 [NGT n = 92; IGT/T2D n = 15]; Men n = 92 [NGT n = 71; IGT/T2D n = 21].
CFH: All subjects n = 279 [NGT n = 243; IGT/T2D n = 36]; Women n = 161 [NGT n = 146; IGT/T2D n = 15]; Men n = 118 [NGT n = 97; IGT/T2D n = 21].

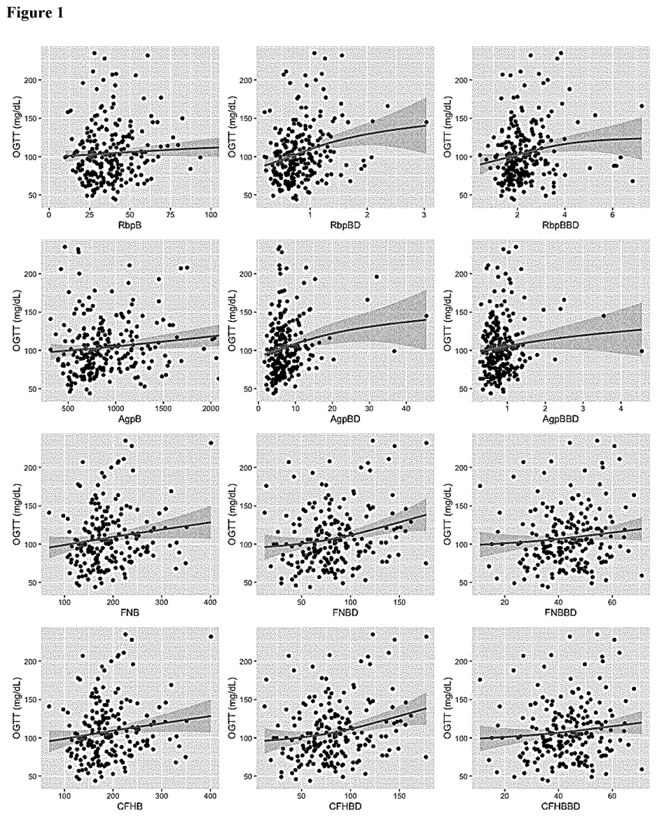

Next, we studied the relationship of the four protein biomarkers (total and bound to GAG) with glucose levels at 2-h PG as a continuous variable (FIG. 1). The Rbp bound to GAG (total and in percentage) increases as glucose levels increase while Rbp levels remain unchanged. A similar trend was observed between the levels of Agp and glucose at 2-h PG. The levels of FN and CFH (total and bound to GAGs) increase as glucose levels at 2-h PG increase, while the percentage remains unchanged.

The levels of glucose at 2-h PG were positively correlated (Table 7) with BMI, FPG, A1C, insulin, Rpb/GAG-bound, Agp/GAG-bound and CFH/GAG-bound for all subjects and stratified by sex. The fasting plasma glucose showed the strongest correlation with glucose levels at 2-h PG for all subjects (0.445, p=0.000). This association was also observed when stratified by sex, with women showing a stronger correlation (0.510, p=0.000) than men (0.363, p=0.000). Both Rbp and CFH bound to GAGs showed a similar correlation with glucose levels at 2-h PG in all subjects (0.26, p=0.000) and in women (0.23, p<0.005). In men, Rbp/GAG-bound showed a higher correlation (0.317, p=0.009) than CFH/GAG-bound (0.286, p=0.006). The correlation between Agp bound to GAGs and glucose levels was lower in all subjects (0.197, p=0.002) as well as in women (0.173, p=0.042) and men (0.221, p=0.028) compared to both Rbp and CFH bound to GAGs. The binding of Rbp, Agp and CFH to GAG suggests a potential opportunity to gain insight into the mechanism of glucose dysmetabolism, but further studies are needed.

It is known that proportions of individual GAG change during development and ageing processes. Thus, it appears that aging might affect the bound of GAG to proteins. In our study, we found a positive correlation between age and FPG, A1C and Rbp, as shown in Table 8. It is important to note that this correlation was observed in all subjects, regardless of sex. A1C showed the strongest correlation with age for all subjects (0.377, p=0.000). This association was also observed when stratified by sex, with women showing a stronger correlation (0.425 p=0.000) than men (0.303, p=0.000). Age appears to be a significant factor for women, as it shows a strong correlations with almost all of the analyzed parameters (Table 8). In contrast, for men, a significant correlation is only observed with FPG (0.293, p=0.003), A1C (0.303, p=0.000), and Rpb (0.197, p=0.013). In women, the highest correlations after A1C were found for Rbp bound to GAGs (0.403, p=0.000), Rbp (0.387, p=0.006), and BMI (0.386, p=0.000). Additionally, both FN bound to GAGs and % FN/GAG-bound showed high correlations (0.368 and 0.364, respectively). A positive correlation was also found with 2-h PG glucose levels (0.295, p=0.001).

TABLE 7
Correlation between serum glucose levels at 2-h PG and various
baseline parameters for all subjects and stratified by sex.

| | All | | Women | | Men | |
|---|---|---|---|---|---|---|
| | Spearman's | | Spearman's | | Spearman's | |
| | rho | p-value | rho | p-value | rho | p-value |
| Age, years | 0.264 | 0.000 | 0.295 | 0.001 | 0.221 | 0.069 |
| BMI, kg/m² | 0.307 | 0.000 | 0.381 | 0.000 | 0.222 | 0.005 |
| FPG, mg/dL | 0.445 | 0.000 | 0.510 | 0.000 | 0.363 | 0.000 |
| A1C, % | 0.318 | 0.000 | 0.360 | 0.000 | 0.261 | 0.002 |
| Fructosamine, umol/L | 0.086 | 0.025 | 0.052 | 0.042 | 0.155 | 0.190 |
| Insulin, mUI/L | 0.225 | 0.000 | 0.188 | 0.008 | 0.270 | 0.000 |
| Rbp, μg/ml | 0.138 | 0.161 | 0.145 | 0.341 | 0.112 | 0.407 |
| Rbp/GAG-bound, μg/ml | 0.262 | 0.000 | 0.233 | 0.004 | 0.317 | 0.009 |
| Rbp/GAG-bound, % | 0.190 | 0.006 | 0.138 | 0.095 | 0.235 | 0.043 |
| Agp, μg/ml | 0.171 | 0.007 | 0.184 | 0.071 | 0.133 | 0.088 |
| Agp/GAG-bound, μg/ml | 0.197 | 0.002 | 0.173 | 0.042 | 0.221 | 0.028 |
| Agp/GAG-bound, % | 0.085 | 0.050 | 0.086 | 0.184 | 0.094 | 0.113 |
| FN, μg/ml | 0.173 | 0.015 | 0.116 | 0.700 | 0.263 | 0.004 |
| FN/GAG-bound, μg/ml | 0.206 | 0.001 | 0.140 | 0.126 | 0.298 | 0.002 |
| FN/GAG-bound, % | 0.143 | 0.052 | 0.127 | 0.178 | 0.154 | 0.154 |
| CFH, μg/ml | 0.240 | 0.797 | 0.176 | 0.338 | 0.341 | 0.942 |
| CFH/GAG-bound, μg/ml | 0.260 | 0.000 | 0.239 | 0.001 | 0.286 | 0.006 |
| CFH/GAG-bound, % | 0.005 | 0.856 | 0.035 | 0.481 | −0.031 | 0.718 |

FPG, fasting plasma glucose; BMI, body mass index; A1C, glycosilated hemoglobin A1c; Rbp, retinol-binding protein 4; Agp, alpha-1-acid glycoprotein; FN, fibronectin; CFH, complement factor H; GAG, glycosaminoglycans; 2-h PG, serum glucose levels (mg/dL) 2 hours after an oral intake of 75 g of glucose. Age, BMI, FPG, A1C, Fructosamine, and Insulin: All subjects n = 280 [NGT n = 244; IGT/T2D n = 36]; Women n = 162 [NGT n = 147; IGT/T2D n = 15]; Men n = 118 [NGT n = 97; IGT/T2D n = 21]. Rbp and Agp: All subjects n = 239 [NGT n = 203; IGT/T2D n = 36]; Women n = 147 [NGT n = 132; IGT/T2D n = 15]; Men n = 92 [NGT n = 71; IGT/T2D n = 21]. FN: All subjects n = 199 [NGT n = 163; IGT/T2D n = 36]; Women n = 107 [NGT n = 92; IGT/T2D n = 15]; Men n = 92 Men [NGT n = 71; IGT/T2D n = 21]. CFH: All subjects n = 279 [NGT n = 243; IGT/T2D n = 36]; Women n = 161 [NGT n = 146; IGT/T2D n = 15]; Men n = 118 [NGT n = 97; IGT/T2D n = 21].

TABLE 8
Correlation between age and several baseline parameters
for all subjects and stratified by sex.

| | All | | Women | | Men | |
|---|---|---|---|---|---|---|
| | Spearman's | | Spearman's | | Spearman's | |
| | rho | p-value | rho | p-value | rho | p-value |
| BMI, kg/m² | 0.279 | 0.000 | 0.386 | 0.000 | 0.170 | 0.198 |
| FPG, mg/dL | 0.278 | 0.000 | 0.282 | 0.004 | 0.293 | 0.003 |
| 2-h PG, mg/dL | 0.264 | 0.000 | 0.295 | 0.001 | 0.221 | 0.069 |
| A1C, % | 0.377 | 0.000 | 0.425 | 0.000 | 0.303 | 0.000 |
| Fructosamine, (μmol/L) | 0.091 | 0.216 | 0.161 | 0.157 | −0.009 | 0.988 |
| Insulin, mUI/L | −0.002 | 0.763 | 0.085 | 0.474 | −0.112 | 0.424 |
| Rbp, μg/ml | 0.259 | 0.001 | 0.387 | 0.006 | 0.197 | 0.013 |
| Rbp/GAG-bound, μg/ml | 0.195 | 0.020 | 0.403 | 0.000 | 0.006 | 0.861 |
| Rbp/GAG-bound, % | −0.014 | 0.486 | 0.080 | 0.872 | −0.141 | 0.243 |
| Agp, μg/ml | 0.144 | 0.024 | 0.204 | 0.023 | 0.065 | 0.194 |
| Agp/GAG-bound, μg/ml | 0.096 | 0.325 | 0.164 | 0.166 | −0.008 | 0.941 |
| Agp/GAG-bound, % | −0.043 | 0.752 | 0.007 | 0.766 | −0.146 | 0.290 |
| FN, μg/ml | 0.089 | 0.349 | 0.174 | 0.040 | 0.041 | 0.946 |
| FN/GAG-bound, μg/ml | 0.221 | 0.004 | 0.368 | 0.000 | 0.107 | 0.433 |
| FN/GAG-bound, % | 0.250 | 0.000 | 0.364 | 0.000 | 0.124 | 0.274 |
| CFH, μg/ml | 0.160 | 0.168 | 0.296 | 0.002 | −0.003 | 0.345 |
| CFH/GAG-bound, μg/ml | 0.176 | 0.029 | 0.214 | 0.056 | 0.128 | 0.206 |
| CFH/GAG-bound, % | −0.046 | 0.598 | −0.089 | 0.175 | 0.003 | 0.521 |

FPG, fasting plasma glucose; BMI, body mass index; A1C, glycosilated hemoglobin A1c; Rbp, retinol-binding protein 4; Agp, alpha-1-acid glycoprotein; FN, fibronectin; CFH, complement factor H; GAG, glycosaminoglycans; 2-h PG, serum glucose levels (mg/dL) 2 hours after an oral intake of 75 g of glucose. BMI, FPG, A1C, Fructosamine, and Insulin: All subjects n = 280 [NGT n = 244; IGT/T2D n = 36]; Women n = 162 [NGT n = 147; IGT/T2D n = 15]; Men n = 118 [NGT n = 97; IGT/T2D n = 21]. Rbp and Agp: All subjects n = 239 [NGT n = 203; IGT/T2D n = 36]; Women n = 147 [NGT n = 132; IGT/T2D n = 15]; Men n = 92 [NGT n = 71; IGT/T2D n = 21]. FN: All subjects n = 199 [NGT n = 163; IGT/T2D n = 36]; Women n = 107 [NGT n = 92; IGT/T2D n = 15]; Men n = 92 Men [NGT n = 71; IGT/T2D n = 21]. CFH: All subjects n = 279 [NGT n = 243; IGT/T2D n = 36]; Women n = 161 [NGT n = 146; IGT/T2D n = 15]; Men n = 118 [NGT n = 97; IGT/T2D n = 21].

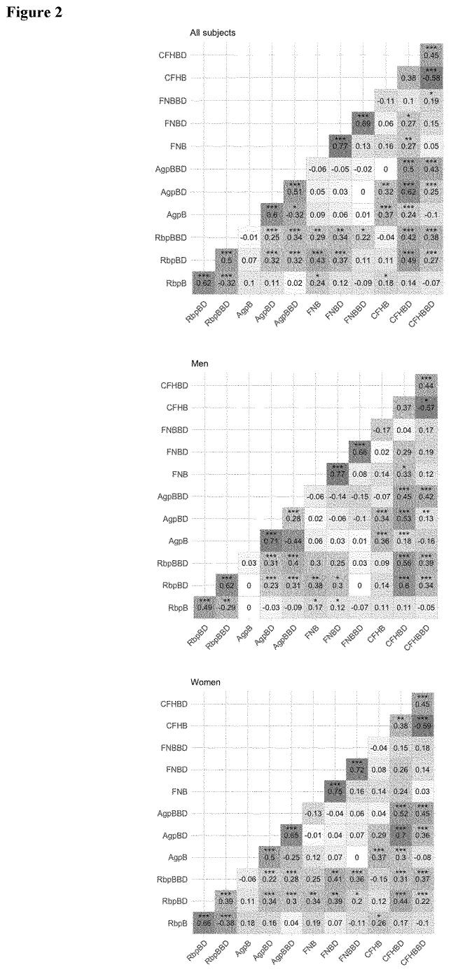

FIG. 2 summarizes the correlation between the four protein biomarkers (total and bound to GAG) tested. A significant positive correlation was observed between total protein (Rbp, Agp and FN) and protein bound to GAG in all subjects, as well as in women and men. Additionally, a positive significant relationship was observed between protein-bound to GAG and the percentage of protein-bound to GAGs for Rbp, Agp, and FN. A significant negative correlation was observed between Rbp and % Rbp/GAG-bound as well in CFH and % CFH/GAG-bound in all subjects, as well as in women and men.

Our results show that the deterioration of glycemic status (measured by OGTT) is related to the binding of GAG to Rbp, Agp, FN and CFH proteins. However, they do not provide information about the type of GAG (HS, CS, DS, KS) to which they bind. A pilot assay was performed to evaluate the binding of Agp and Rbp to increasing amounts of CS, HS, and HA (Table 9). It was found that Rbp has a higher affinity for CS (878±146) compared to HS (313±66) and HA (271±40). For Agp, however, low concentrations of the GAGs tested interfere with binding (CS 69±18, HS 53±15, and HA 55±15). Only in the case of CS is there an increase in binding at the highest concentration (173±57). Further studies are necessary for the understanding of the relationships between the different GAGs and proteins.

TABLE 9
Binding of GAGs to Agp and Rbp

| | Agp/GAG-bound | Rbp/GAG-bound |
|---|---|---|
| Serum | 100.00 ± 13.20 | 100.00 ± 15.01 |
| Serum + CS 0.5 μg | 69.66 ± 18.06 | 91.44 ± 30.90 |
| Serum + CS 1.0 μg | 63.00 ± 7.16 | 99.71 ± 21.00 |
| Serum + CS 5.0 μg | 99.33 ± 21.71 | 337.19 ± 36.19 |
| Serum + CS 10.0 μg | 118.81 ± 11.50 | 618.40 ± 78.06 |
| Serum + CS 15.0 μg | 173.81 ± 57.72 | 878.12 ± 146.37 |
| Serum + HS 0.5 μg | 53.49 ± 14.98 | 69.11 ± 8.39 |
| Serum + HS 1.0 μg | 49.97 ± 17.31 | 61.45 ± 10.13 |
| Serum + HS 5.0 μg | 81.76 ± 14.28 | 134.21 ± 3.10 |
| Serum + HS 10.0 μg | 98.64 ± 19.70 | 195.32 ± 32.02 |
| Serum + HS 15.0 μg | 107.08 ± 8.99 | 313.23 ± 66.46 |
| Serum + HA 0.5 μg | 55.40 ± 15.84 | 77.55 ± 20.55 |
| Serum + HA 1.0 μg | 44.88 ± 22.16 | 56.43 ± 8.59 |
| Serum + HA 5.0 μg | 81.21 ± 5.90 | 123.59 ± 5.00 |
| Serum + HA 10.0 μg | 62.05 ± 11.59 | 171.39 ± 12.31 |
| Serum + HA 15.0 μg | 84.21 ± 12.38 | 271.29 ± 40.19 |

Agp, alpha-1-acid glycoprotein;
Rbp, retinol-binding protein 4;
CS, Chondroitin sulfate;
HS, Heparan sulfate;
HA, Hyaluronic acid;
μg, micrograms.
Data are expressed as mean ± SD.
The percentages of Agp and Rbp bound to GAGs were calculated relative to serum with not added GAGs, which was ser as 100%..

These results may suggest a clue to identify novel pathways related to the glucose dysmetabolism. It is known that diabetes is accompanied by increased proteolytic in various tissues. This may lead to the cleavage of protein cores of proteoglycans releasing the GAG from these complexes. The products of proteoglycan degradation may penetrate into circulating blood and elevate the concentration of GAG in the serum. Thus, free GAGs could bind to the proteins present in the serum and cause a change in their function. This opens a new line of research on the effects of glucose on the binding of glycosaminoglycans to proteins with possible applications in the diagnosis, monitoring, prognosis and discovery of new therapeutic targets in prediabetes and/or IGT/T2D.

### Example 2.4. Prediction of 2-h Glucose Levels after OGTT

We performed a stepwise logistic regression analysis including FPG, Rbp (total and bound to GAG), Agp (total and bound to GAG), FN (total and bound to GAG) and CFH (total and bound to GAG) to predict IGT/T2D (Table 10). The models that include FPG showed an increase in R² and discrimination capacity (AUC). The best performance was observed in the IGT/T2D diagnostic model with FPG+Rbp/GAG-bound (AUC 0.882) for all subjects and women (AUC 0.886). In both men and women, FPG+CFH/GAG-bound exhibited excellent performance with an AUC of 0.949 and 0.866, respectively. Note that in women, although FPG+Rbp has a similar AUC to FPG+Rbp/GAG-bound (0.889 vs. 0.886), the R² is higher in the model that includes GAG-bound (0.339 vs. 0.365).

Further, to identify the best predictive performance across the 2-h PG spectrum of glycemia after an OGTT, we tested different combinations of the four biomarkers (total and GAG-bound) and clinical parameters using generalized additive models (GAM). Table 11 displays the models with the best performance for all subjects and categorized according to sex. The highest R² value (0.393) was observed when the response variable was the glucose levels at 2-h PG, and the predictors were FPG, FN/GAG-bound and CFH/GAG-bound in men, and for all subjects the R² was 0.334. Age is an important factor in women (as shown in Table 8), and the highest R² value (0.293) was observed using the glucose levels at 2-h PG as response variable and FPG, age, Rbp/GAG-bound or Agp/GAG-bound as predictors.

TABLE 10
Logistic regression models using the four biomarkers (total
and GAG-bound), FPG, and their combination to predict
IGT/T2D for all subjects and stratified by sex.

| IGT/T2D | All | | Men | | Women | |
|---|---|---|---|---|---|---|
| 2-h PG > 139 mg/dL | R² | AUC | R² | AUC | R² | AUC |
| FPG | 0.194 | 0.819 | 0.161 | 0.845 | 0.261 | 0.796 |
| Rbp | 0.010 | 0.601 | 0.007 | 0.589 | 0.010 | 0.575 |
| FPG + Rbp | 0.219 | 0.837 | 0.195 | 0.854 | 0.339 | 0.889 |
| Rbp/GAG-bound | 0.096 | 0.725 | 0.092 | 0.729 | 0.088 | 0.706 |
| FPG + Rbp/GAG-bound | 0.280 | 0.882 | 0.207 | 0.849 | 0.365 | 0.886 |
| % Rbp GAG-bound | 0.065 | 0.703 | 0.036 | 0.631 | 0.098 | 0.756 |
| FPG + % Rbp GAG-bound | 0.228 | 0.836 | 0.210 | 0.849 | 0.287 | 0.817 |
| Agp | 0.037 | 0.678 | 0.022 | 0.673 | 0.010 | 0.500 |
| FPG + Agp | 0.264 | 0.854 | 0.213 | 0.863 | 0.303 | 0.817 |
| Agp/GAG-bound | 0.110 | 0.754 | 0.084 | 0.756 | 0.150 | 0.820 |
| FPG + Agp/GAG-bound | 0.251 | 0.846 | 0.241 | 0.891 | 0.356 | 0.859 |
| % Agp/GAG-bound | 0.051 | 0.605 | 0.131 | 0.666 | 0.045 | 0.630 |
| FPG + % Agp/GAG-bound | 0.256 | 0.848 | 0.263 | 0.859 | 0.318 | 0.849 |
| FN | 0.035 | 0.667 | 0.002 | 0.536 | 0.033 | 0.631 |
| FPG + FN | 0.215 | 0.823 | 0.238 | 0.873 | 0.283 | 0.814 |
| FN/GAG-bound | 0.024 | 0.588 | 0.130 | 0.772 | 0.018 | 0.606 |
| FPG + FN/GAG-bound | 0.250 | 0.852 | 0.268 | 0.918 | 0.281 | 0.812 |
| % FN/GAG-bound | 0.012 | 0.619 | 0.034 | 0.641 | 0.009 | 0.620 |
| FPG + % FN/GAG-bound | 0.228 | 0.840 | 0.241 | 0.879 | 0.281 | 0.818 |
| CFH | 0.026 | 0.643 | 0.020 | 0.698 | 0.064 | 0.695 |
| FPG + CFH | 0.197 | 0.816 | 0.368 | 0.935 | 0.280 | 0.824 |
| CFH/GAG-bound | 0.044 | 0.670 | 0.088 | 0.696 | 0.097 | 0.708 |
| FPG + CFH/GAG-bound | 0.213 | 0.831 | 0.457 | 0.949 | 0.382 | 0.866 |
| % CFH/GAG-bound | 0.001 | 0.560 | 0.002 | 0.587 | 0.001 | 0.574 |
| FPG + % CFH/GAG-bound | 0.198 | 0.817 | 0.293 | 0.913 | 0.260 | 0.800 |

FPG, fasting plasma glucose; Rbp, retinol-binding protein 4; Agp, alpha-1-acid glycoprotein; FN, fibronectin; CFH, complement factor H; GAG, glycosaminoglycans; 2-h PG, serum glucose levels (mg/dL) 2 hours after an oral intake of 75 g of glucose; IGT, impaired glucose tolerance; T2D, type 2 diabetes

TABLE 11
Generalized Additive Models (GAM) using the four biomarkers (total and
GAG-bound), FPG, and their combination to predict glucose levels at 2-h
PG for all subjects and stratified by sex.

| | | Deviance |
|---|---|---|
| | R² | explained |
| All subjects | | |
| 2-h PG~FPG | 0.244 | 24.6 |
| 2-h PG~FPG + FN/GAG-bound + CFH/GAG-bound | 0.334 | 34.4 |
| Men | | |
| 2-h PG~FPG | 0.223 | 23.0 |
| 2-h PG~FPG + FN/GAG-bound + CFH/GAG-bound | 0.393 | 41.3 |
| Women | | |
| 2-h PG~FPG | 0.261 | 26.6 |
| 2-h PG~Age + FPG | 0.276 | 28.5 |
| 2-h PG~Age + FPG + Rbp/GAG-bound | 0.293 | 30.7 |
| 2-h PG~Age + FPG + Agp/GAG-bound | 0.293 | 30.7 |

FPG, fasting plasma glucose;
Rbp, retinol-binding protein 4;
Agp, alpha-1-acid glycoprotein;
FN, fibronectin;
CFH, complement factor H;
GAG, glycosaminoglycans;
2-h PG, serum glucose levels (mg/dL) 2 hours after an oral intake of 75 g of glucose.

### Example 2.5. Prediction of Prediabetes

The clinical characteristics of the 279 individuals analyzed according to their basal glycemic status (normoglycemia and prediabetes) are shown in Table 12. As expected, as the glycemic status worsens (from normoglycemia to prediabetes), there was a significant increase in age, BMI, FPG, A1C, fructosamine and insulin. In general, no differences were found between men and woman.

TABLE 12
Clinical characteristics of the individuals analyzed according to basal
glycemic status for all subjects and stratified by sex.

| | | Normoglycemia | Prediabetes | p-value |
|---|---|---|---|---|
| Age | Men | 53.70 ± 14.58 | 61.65 ± 12.80 | 0.010 |
| (years) | Women | 53.07 ± 15.16 | 63.94 ± 12.36 | <0.001 |
| | All | 53.35 ± 14.87 | 63.10 ± 12.50 | <0.001 |
| BMI | Men | 28.53 ± 4.11 | 31.32 ± 4.63 | 0.004 |
| (kg/m²) | Women | 27.39 ± 4.96 | 30.52 ± 5.44 | <0.001 |
| | All | 27.90 ± 4.62 | 30.82 ± 5.14 | <0.001 |
| FPG | Men | 84.57 ± 7.85 | 99.77 ± 11.29 | <0.001 |
| (mg/dL) | Women | 81.83 ± 7.99 | 92.30 ± 12.16 | <0.001 |
| | All | 83.06 ± 8.02 | 95.06 ± 12.32 | <0.001 |
| A1C | Men | 5.30 ± 0.24 | 5.81 ± 0.25 | <0.001 |
| (%) | Women | 5.25 ± 0.28 | 5.81 ± 0.26 | <0.001 |
| | All | 5.28 ± 0.26 | 5.81 ± 0.25 | <0.001 |
| Fructosamine | Men | 213.60 ± 28.76 | 224.39 ± 32.56 | 0.121 |
| (μmol/L) | Women | 220.78 ± 31.36 | 236.02 ± 34.70 | 0.004 |
| | All | 217.56 ± 30.36 | 231.73 ± 34.20 | <0.001 |
| Insulin | Men | 9.19 ± 7.29 | 13.78 ± 10.28 | 0.016 |
| (mUI/L) | Women | 8.61 ± 4.55 | 10.56 ± 4.92 | 0.006 |
| | All | 8.88 ± 5.96 | 11.75 ± 7.47 | <0.001 |

FPG, fasting plasma glucose;
BMI, body mass index;
A1C, glycosilated hemoglobin A1c.
Data are expressed as mean + SD.
The p-values were performed using Mann-Whitney test.
All subjects n = 279 [Normoglucemia n = 193; Prediabetes n = 86]; Women n = 161 [Normoglucemia n = 106; Prediabetes n = 55]; Men n = 118 [Normoglucemia n = 87; Prediabetes n = 31].

Table 13 shows the basal serum levels of the four selected biomarkers (total and GAG-bound) in individuals classified as normoglycemia and prediabetes in all subjects and stratified by sex.

The levels of Rbp increased (29.00 vs 38.31 μg/ml, p=0.029) as moved from normoglycemia to prediabetes for women. Rbp levels were lower in women than in men (29.00 vs 38.61 μg/ml, p<0.001) in the normoglycemia group. The levels of Rbp/GAG-bound increased significantly as moved from normoglycemia to prediabetes in all subjects (0.71 vs 0.86 μg/ml, p=0.016) and in men (0.81 vs 1.12 μg/ml p=0.021). Again, the Rbp/GAG-bound levels were lower in women than in men (normoglycemia 0.65 vs 0.81 μg/ml, prediabetes 0.74 vs 1.12 μg/ml respectively, p<0.001). No differences were observed in the levels of the percentage of Rbp/GAG bound.

Agp levels increased (772.83 vs 912.36 μg/ml, p=0.002) as women progressed from normoglycemia to prediabetes. Agp levels were lower in women than in men (772.83 vs 851.48 μg/ml, p<0.05) in the normoglycemia. The levels of Rbp/GAG-bound increased significantly from normoglycemia to prediabetes in all subjects (6.00 vs 7.00 μg/ml, p=0.008) and in women (5.78 vs 7.21 μg/ml, p=0.001). No differences were observed in the levels of the percentage of Agp/GAG bound.

FN concentrations increased significantly from normoglycaemia to prediabetes in all subjects (171.41 vs 205.40 μg/ml, p<0.001) and in women (163.21 vs 193.62 μg/ml, p<0.001). In the normoglycaemic group, FN levels were lower in women than in men (163.21 vs 190.45 μg/ml, p<0.001). Similarly, FN/GAG-bound concentrations increased significantly from normoglycemia to prediabetes in all subjects (75.56 vs 86.25 μg/ml, p=0.009) and in women (66.70 vs 84.50 μg/ml p<0.001). In the normoglycaemic group, FN/GAG-bound levels were lower in women than in men (66.70 vs. 84.19 μg/ml, p<0.001). No differences were observed in the levels of the percentage of FN/GAG bound in any of the groups tested.

In the levels of CFH, a significant increase was observed as moved from a state of normoglycemia to prediabetes in all subjects (48.22 vs 57.26 μg/ml, p<0.001) and in women (45.20 vs 58.27 μg/ml, p<0.001). The levels of CFH/GAG-bound increased significantly as moved from normoglycemia to prediabetes in all subjects (0.54 vs 0.62 μg/ml, p<0.001) and in women (0.52 vs 0.63 μg/ml, p<0.001). No differences were observed in the levels of % CFH bound to GAG in any of the groups tested.

We performed a stepwise logistic regression analysis including FPG and the four biomarkers (total and bound to GAGs) to diagnosis of prediabetes (Table 14). An increase in R² and discriminatory capacity (AUC) was observed in the models including FPG. In women, the prediabetes diagnostic model with FPG+% Agp/GAG-bound (AUC 0.997) had the best performance. Note that FPG+Agp/GAG-bound performed well in women (AUC 0.989) and is the easiest to use. The prediabetes diagnostic model with FPG+FN/GAG-bound showed excellent performance in men (AUC 0.894) and all subjects (AUC 0.869).

TABLE 13
Basal serum levels of the four biomarkers (total and GAG-bound) according to
basal glycemic status for all subjects and stratified by sex.

| | | Normoglycemia | Prediabetes | p-value |
|---|---|---|---|---|
| Rbp | Men | 38.61 [11.52-109.96] | 40.63 [19.25-109.21] | 0.490 |
| (ug/ml) | Women | 29.00 [9.89-93.51]* | 38.31 [13.31-272.15] | 0.029 |
| | All | 33.69 [9.89-109.96] | 39.06 [13.31-272.15] | 0.070 |
| Rbp/GAG-bound | Men | 0.81 [0.20-3.04] | 1.12 [0.47-2.10] | 0.021 |
| (ug/ml) | Women | 0.65 [0.20-2.08] | 0.74 [0.29-2.36]** | 0.092 |
| | All | 0.71 [0.20-3.04] | 0.86 [0.29-2.36] | 0.016 |
| Rbp GAG-bound | Men | 2.00 [0.70-6.15] | 2.47 [1.35-6.06] | 0.058 |
| (%) | Women | 2.09 [0.87-6.85] | 2.13 [0.43-7.23] | 0.832 |
| | All | 2.07 [0.70-6.85] | 2.15 [0.43-7.23]* | 0.363 |
| Agp | Men | 851.48 [315.42-4230.73] | 740.40 [446.25-2056.69] | 0.490 |
| (μg/ml) | Women | 772.83 [393.06-1561.87]* | 912.36 [314.47-2082.00] | 0.002 |
| | All | 801.84 [315.42-4230.73] | 867.94 [314.47-2082.00] | 0.068 |
| Agp/GAG-bound | Men | 6.38 [2.32-45.45] | 6.37 [3.16-13.70] | 0.784 |
| (μg/ml) | Women | 5.78 [1.64-36.78] | 7.21 [2.25-29.49] | 0.001 |
| | All | 6.00 [1.64-45.45]* | 7.00 [2.25-29.49] | 0.008 |
| Agp/GAG-bound | Men | 0.77 [0.36-3.55] | 0.77 [0.52-1.45] | 0.535 |
| (%) | Women | 0.74 [0.28-4.55] | 0.90 [0.28-2.50] | 0.142 |
| | All | 0.76 [0.28-4.55] | 0.85 [0.28-2.50] | 0.106 |
| FN | Men | 190.45 [110.99-335.90] | 214.09 [110.05-401.10] | 0.148 |
| (μg/ml) | Women | 163.21 [97.45-282.54]** | 193.62 [68.53-349.20] | <0.001 |
| | All | 171.41 [97.45-335.90] | 205.40 [68.53-401.10] | <0.001 |
| FN/GAG-bound | Men | 84.19 [18.76-161.71] | 91.09 [42.30-177.31] | 0.596 |
| (μg/ml) | Women | 66.70 [14.22-116.28]*** | 84.50 [12.41-176.90] | <0.001 |
| | All | 75.56 [14.22-161.71] | 86.25 [12.41-177.31] | 0.009 |
| FN/GAG-bound | Men | 45.16 [14.10-64.36] | 39.63 [23.25-65.02] | 0.204 |
| (%) | Women | 40.55 [10.78-70.97] | 44.49 [17.42-68.03] | 0.182 |
| | All | 42.75 [10.78-70.97] | 42.44 [17.42-68.03] | 0.914 |
| CFH | Men | 50.23 [17.11-2268.38] | 56.26 [21.12-125.89] | 0.332 |
| (μg/ml) | Women | 45.20 [14.11-186.85] | 58.27 [31.15-222.58] | <0.001 |
| | All | 48.22 [14.11-2268.38] | 57.26 [21.12-222.58] | <0.001 |
| CFH/GAG-bound | Men | 0.55 [0.30-2.33] | 0.62 [0.30-1.15] | 0.148 |
| (μg/ml) | Women | 0.52 [0.25-1.62] | 0.63 [0.32-1.27] | <0.001 |
| | All | 0.54 [0.25-2.33] | 0.62 [0.30-1.27] | <0.001 |
| CFH/GAG-bound | Men | 1.11 [0.03-4.21] | 1.06 [0.54-3.66] | 0.856 |
| (%) | Women | 1.21 [0.24-4.14] | 1.13 [0.28-2.59] | 0.403 |
| | All | 1.18 [0.03-4.21] | 1.10 [0.28-3.66] | 0.414 |

Rbp, retinol-binding protein 4;
Agp, alpha-1-acid glycoprotein;
FN, fibronectin;
CFH, complement factor H;
GAG, glycosaminoglycans.
Data are expressed as median [min-max].
p-value: *< 0.05, **< 0.01, ***< 0.001 men vs women.
The p-values were performed using Mann-Whitney test.
Rbp and Agp: All subjects n = 239 [Normoglucemia n = 166; Prediabetes n = 73]; Women n = 147 [Normoglucemia n =100; Prediabetes n = 47]; Men n = 92 [Normoglucemia n = 66; Prediabetes n = 26].
FN: All subjects n = 199 [Normoglucemia n = 137; Prediabetes n = 62]; Women n = 107 [Normoglucemia n = 71; Prediabetes n = 36]; Men n = 92 [Normoglucemia n = 66; Prediabetes n = 26].
CFH: All subjects n = 279 [Normoglucemia n = 193; Prediabetes n = 86]; Women n = 161 [Normoglucemia n = 106; Prediabetes n = 55]; Men n = 118 [Normoglucemia n = 87; Prediabetes n = 31].

TABLE 14
Logistic regression models using the four biomarkers (total
and GAG-bound), FPG, and their combination to predict
prediabetes for all subjects and stratified by sex.

| | All | | Men | | Women | |
|---|---|---|---|---|---|---|
| | R² | AUC | R² | AUC | R² | AUC |
| FPG | 0.330 | 0.813 | 0.268 | 0.809 | 0.526 | 0.867 |
| Rbp | 0.012 | 0.577 | 0.082 | 0.699 | 0.001 | 0.546 |
| FPG + Rbp | 0.336 | 0.817 | 0.290 | 0.810 | 0.543 | 0.898 |
| Rbp/GAG-bound | 0.022 | 0.604 | 0.014 | 0.599 | 0.050 | 0.656 |
| FPG + Rbp/GAG-bound | 0.329 | 0.820 | 0.241 | 0.794 | 0.543 | 0.900 |
| % Rbp GAG-bound | 0.001 | 0.547 | 0.007 | 0.506 | 0.017 | 0.628 |
| FPG + % Rbp GAG-bound | 0.334 | 0.821 | 0.247 | 0.796 | 0.542 | 0.901 |
| Agp | 0.015 | 0.575 | 0.090 | 0.653 | 0.007 | 0.546 |
| FPG + Agp | 0.349 | 0.824 | 0.308 | 0.825 | 0.541 | 0.901 |
| Agp/GAG-bound | 0.066 | 0.622 | 0.114 | 0.670 | 0.050 | 0.662 |
| FPG + Agp/GAG-bound | 0.371 | 0.858 | 0.317 | 0.856 | 0.822 | 0.989 |
| % Agp/GAG-bound | 0.035 | 0.619 | 0.055 | 0.663 | 0.014 | 0.602 |
| FPG + % Agp/GAG-bound | 0.357 | 0.854 | 0.288 | 0.841 | 0.877 | 0.997 |
| FN | 0.067 | 0.668 | 0.165 | 0.726 | 0.026 | 0.597 |
| FPG + FN | 0.430 | 0.866 | 0.412 | 0.864 | 0.557 | 0.900 |
| FN/GAG-bound | 0.057 | 0.620 | 0.214 | 0.767 | 0.011 | 0.601 |
| FPG + FN/GAG-bound | 0.425 | 0.869 | 0.458 | 0.894 | 0.554 | 0.903 |
| % FN/GAG-bound | 0.004 | 0.514 | 0.011 | 0.594 | 0.001 | 0.585 |
| FPG + % FN/GAG-bound | 0.409 | 0.857 | 0.353 | 0.861 | 0.560 | 0.912 |
| CFH | 0.043 | 0.641 | 0.103 | 0.713 | 0.006 | 0.570 |
| FPG + CFH | 0.349 | 0.834 | 0.315 | 0.842 | 0.523 | 0.867 |
| CFH/GAG-bound | 0.066 | 0.672 | 0.105 | 0.707 | 0.016 | 0.621 |
| FPG + CFH/GAG-bound | 0.343 | 0.833 | 0.312 | 0.840 | 0.523 | 0.865 |
| % CFH/GAG-bound | 0.002 | 0.523 | 0.038 | 0.659 | 0.008 | 0.511 |
| FPG + % CFH/GAG-bound | 0.330 | 0.825 | 0.304 | 0.838 | 0.522 | 0.870 |

FPG, fasting plasma glucose; Rbp, retinol-binding protein 4; Agp, alpha-1-acid glycoprotein; FN, fibronectin; CFH, complement factor H; GAG, glycosaminoglycans.

Key findings are summarized in Table 15. In women, for the diagnosis of IGT/T2D and prediabetes, the determination of Agp/GAG-bound and Rbp/GAG-bound would be useful. In men, biomarkers for the diagnosis of IGT/T2D and prediabetes would be FN/GAG-bound and CFH/GAG-bound.

TABLE 15
Summary of the main findings.

| | |
|---|---|
| All subjects | |
| Prediabetes | FPG + FN/GAG-bound (AUC 0.869) |
| IGT/T2D | FPG + Rbp/GAG-bound (AUC 0.882) |
| Glucose levels at 2-h | FPG + FN/GAG-bound + CFH/GAG-bound |
| PG | (R² 0.334) |
| Men | |
| Prediabetes | FPG + FN/GAG-bound (AUC 0.894) |
| IGT/T2D | FPG + CFH/GAG-bound (AUC 0.949) |
| Glucose levels at 2-h | FPG + FN/GAG-bound + CFH/GAG-bound |
| PG | (R² 0.393) |
| Women | |
| Prediabetes | FPG + Agp/GAG-bound (AUC 0.989) |
| IGT/T2D | FPG + Rbp/GAG-bound (AUC 0.886) |
| Glucose levels at 2-h | Age + FPG + Rbp/GAG-bound (R² 0.293) |
| PG | or |
| | Age + FPG + Agp/GAG-bound (R² 0.293) |

FPG, fasting plasma glucose;
Rbp, retinol-binding protein 4;
Agp, alpha-1-acid glycoprotein;
FN, fibronectin;
CFH, complement factor H;
GAG, glycosaminoglycans;
2-h PG, serum glucose levels (mg/dL) 2 hours after an oral intake of 75 g of glucose;
IGT, impaired glucose tolerance;
T2D, Type 2 diabetes.

1. In vitro method for the diagnosis, monitoring or prognosis of Type 2 diabetes (T2D), impaired glucose tolerance (IGT), or prediabetes which comprises assessing the amount of a protein, selected from the group consisting of: Retinol-Binding Protein 4, Alpha-1-acid glycoprotein, fibronectin and/or complement factor H which is/are bound to glycosaminoglycans, in a biological sample obtained from the subject, wherein the determination of a higher amount of a protein, selected from the group consisting of: Retinol-Binding Protein 4, Alpha-1-acid glycoprotein, fibronectin and/or complement factor H bound to glycosaminoglycans, as compared with a pre-established threshold value determined in subject who are not suffering from diabetes or prediabetes, is an indication that the subject may be suffering from T2D, IGT or prediabetes, or that the subject has a poor prognosis.

2. In vitro method, according to topic 1, characterized in that it is performed once an oral glucose tolerance test (OGTT) is carried out.

3. In vitro method for screening or selecting patient candidates to be subjected to OGTT which comprises assessing the amount of a protein, selected from the group consisting of: Retinol-Binding Protein 4, Alpha-1-acid glycoprotein, fibronectin and/or complement factor H which is/are bound to glycosaminoglycans, in a biological sample obtained from the patient, wherein the determination of a higher amount of a protein, selected from the group consisting of Retinol-Binding Protein 4, Alpha-1-acid glycoprotein, fibronectin and/or complement factor H which is/are bound to glycosaminoglycans as compared with a pre-established threshold value, is an indication that the patient may be subjected to OGTT.

4. In vitro method, according to any of the claims 1 to 3, in combination with the determination of fasting plasma glucose (FPG).

5. In vitro method, according to any of the previous claims, wherein the biological samples is blood, plasma or serum.

6. In vitro use of a protein selected from the group consisting of: Retinol-Binding Protein 4, Alpha-1-acid glycoprotein, fibronectin and/or complement factor H which is/are bound to glycosaminoglycans, for the diagnosis, monitoring or prognosis of T2D, IGT or prediabetes; or for screening or selecting patient candidates to be subjected to OGTT.

7. Immunoassay kit comprising antibodies for determining the amount of a protein selected from the group consisting of: Retinol-Binding Protein 4, Alpha-1-acid glycoprotein, fibronectin and/or complement factor H bound to GAGs.

8. Use of the immunoassay of claim 7 for the diagnosis, monitoring or prognosis of T2D, IGT or prediabetes; or for screening or selecting patient candidates to be subjected to OGTT.
